(19) United States
(12) Patent Application Publication
(10) Pub. No.: US 2022/0139494 A1
(43) Pub. Date: May 5, 2022

(54) REACTION MODELING WITH DYNAMIC SOURCES AND SINKS

(71) Applicant: X Development LLC, Mountain View, CA (US)

(72) Inventor: Frank Russo, Sunnyvale, CA (US)

(21) Appl. No.: 16/949,495

(22) Filed: Oct. 30, 2020

(51) Int. Cl. G16B 5/00

(52) U.S. Cl. CPC G16B 5/00

(57) ABSTRACT

The present disclosure relates to modeling biological systems and biochemical processes. In order to accurately model these systems and processes, the behavior at the boundary of models of the system and processes is important. Some embodiments include representing rates of change of concentrations of molecules at the boundaries of models as dynamic and responsive rather than static and invariant. The rates of change of the concentrations of molecules may be modeled as proportional controllers. In some embodiments, the proportional controllers may be saturable. Using responsive boundaries reduces model complexity, thereby increasing computational speed and efficiency. Additionally, the responsive boundaries may more accurately and realistically depict the behavior of components within a system compared to other boundary modeling techniques. Alternatively or additionally, some embodiments may include using a result of a model with responsive boundaries to engineer or alter a biological system.

### FIELD

The present disclosure relates to kinetic modeling of biochemical pathways, and in particular to general and scalable techniques for modeling in silico the kinetics of systems of connected biochemical reactions.

### BACKGROUND

A biological system such as a living cell, or a population of living cells, can be modeled in silico such that the response of the living cell(s) to a variety of experiments can be performed quickly and cheaply in simulation. Simulated experiments may be performed to develop an understanding of the interrelationships of various environmental and other factors on the development of the biological system and/or on the biological system's effect on its environment. For example, simulated experiments may be performed to determine a set of environmental conditions that increases a growth rate of the biological system or that increases a rate of production of a substance of interest (e.g., an antibody, a hormone, a protein, an enzyme) by the biological system. Additionally or alternatively, the simulated experiments may be performed to develop an understanding of the effect of changes in the composition of the biological system (e.g., changes in the genetic or epigenetic makeup of the biological system) on the development or behavior of the biological system and/or to develop an understanding of the effect on changes in protein structure on the functionality of such proteins. For example, simulated experiments may be performed to determine a change in the genome of the biological system that increases a growth rate of the biological system and/or that increases a rate of production of a substance of interest by the biological system.

Biological systems and the biochemical processes thereof are typically modeled using various equations or functions and include many parameters, each corresponding to (e.g., ‘modeling’) an aspect of the structure and/or function of the biological system or biochemical process. Of the biochemical processes that need to be modeled to both infer molecular mechanisms and predict biological responses, many are enzyme reactions typically described using a system of differential equations. Biological systems and biochemical processes thereof that are modeled may interact with other biological systems and biochemical processes by exchanging mass, energy, and/or information. In order to accurately model biological systems and biochemical processes, the behavior at the boundaries of a biological system should be considered. As levels of components entering or exiting a biological system may be regulated, consumed, or produced by other biological systems, modeling these components as static may not be accurate. Accordingly, there is a need to model boundary components as responsive to changes in the model.

### SUMMARY

Biological systems, including enzymatic reactions, may be represented in kinetic reaction models. The behavior at the boundary of these models is important. Embodiments of the present invention include representing rates of change of concentrations of molecules at the boundaries of models as dynamic and responsive rather than static and invariant. The rates of change of the concentrations of molecules may be modeled as proportional controllers. In some embodiments, the proportional controllers may be saturable. Using responsive boundaries reduces model complexity, thereby increasing computational speed and efficiency. Additionally, the responsive boundaries may more accurately and realistically depict the behavior of components within a system compared to other boundary modeling techniques. Alternatively or additionally, some embodiments may include using a result of a model with responsive boundaries to engineer or alter a biological system.

Some embodiments include a computer-implemented method. The computer-implemented method may include initializing a model of an overall reaction. The model may include a plurality of rate equations. Each rate equation may correspond to an intermediate reaction of the overall reaction. The overall reaction may be part of a pathway or process in a system to be modeled. The plurality of rate equations may include concentrations of molecules. The molecules may include a first molecule. The plurality of rate equations may include a first rate equation. The first rate equation may correspond to a first intermediate reaction of the overall reaction. The first rate equation may include a concentration of the first molecule. A rate of change of the concentration of the first molecule may be configured to depend on a separation value of the concentration from a setpoint. The method may include simulating an in silico behavior of the system. Simulating the behavior may be by generating a plurality of rates of change of the concentrations of molecules using the model of the overall reaction.

In some embodiments, the rate of change of the concentration of the first molecule may be configured to depend on a saturation constant and a proportional constant. The rate of change of the concentration of the first molecule may be configured to approach the product of the saturation constant and the proportional constant as the separation value increases. The rate of change of the concentration of the first molecule may be configured to approach the product of the proportional constant and the separation value as the separation value decreases.

In some embodiments, the saturation constant, the proportional constant, or the setpoint may be adjusted after comparing a generated rate of change of the concentration of the first molecule with a reference rate of change of the concentration of the first molecule.

In some embodiments, the model may be configured such that the concentration of the first molecule is not increased or not decreased in the plurality of rate equations other than in the first rate equation and a second rate equation corresponding to a second intermediate reaction that is the reverse of the first intermediate reaction.

In some embodiments, the model may be configured such that the concentration of the first molecule is not increased in the plurality of rate equations other than the first rate equation.

In some embodiments, the rate of change of the concentration of the first molecule may be configured to be proportional to the separation value.

In some embodiments, the rate of change of the concentration may be represented by

$$\frac{{k}_{p} {k}_{sat} sep}{sep + {k}_{sat}} ,$$

where kₚ is a proportional constant, kₛₐₜ is a saturation constant, and sep is the separation value.

In some embodiments, a system is provided that includes one or more data processors and a non-transitory computer readable storage medium containing instructions which, when executed on the one or more data processors, cause the one or more data processors to perform part or all of one or more methods disclosed herein.

In some embodiments, a computer-program product is provided that is tangibly embodied in a non-transitory machine-readable storage medium and that includes instructions configured to cause one or more data processors to perform part or all of one or more methods disclosed herein.

Some embodiments of the present disclosure include a system including one or more data processors. In some embodiments, the system includes a non-transitory computer readable storage medium containing instructions which, when executed on the one or more data processors, cause the one or more data processors to perform part or all of one or more methods and/or part or all of one or more processes disclosed herein. Some embodiments of the present disclosure include a computer-program product tangibly embodied in a non-transitory machine-readable storage medium, including instructions configured to cause one or more data processors to perform part or all of one or more methods and/or part or all of one or more processes disclosed herein.

The terms and expressions which have been employed are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention as claimed has been specifically disclosed by embodiments and optional features, modification and variation of the concepts herein disclosed may be resorted to by those skilled in the art, and that such modifications and variations are considered to be within the scope of this invention as defined by the appended claims.

### BRIEF DESCRIPTION OF THE DRAWINGS

The present invention will be better understood in view of the following non-limiting figures, in which:

FIG. 1 shows an interaction system for configuring and using a simulation to facilitate subsequent experiment configurations according to various embodiments;

FIG. 2 shows a representation of modules representing distinct biological functions according to various embodiments;

FIG. 3 shows a simulation controller that dynamically integrates results generated by different types of models to simulate higher-level states and reactions according to various embodiments;

FIG. 4 shows a process for dynamically synthesizing results generated by multiple simulators to simulate higher-level results according to various embodiments;

FIG. 5 shows a module-specific simulation controller to simulate states and reactions according to various embodiments;

FIG. 6 shows a process for using a simulator to generate metabolite time-course data according to various embodiments;

FIG. 7 illustrate the production of the amino acid threonine according to various embodiments;

FIGS. 8A and 8B illustrate an enzymology assay according to various embodiments;

FIGS. 9A, 9B, 9C, and 9D illustrate pathway from mannose-6P to GDP-mannose and different system boundaries according to various embodiments;

FIG. 10 shows methods for modeling a reaction according to various embodiments;

FIG. 11 illustrates the last two steps of threonine synthesis and possible system boundaries according to various embodiments;

FIGS. 12A, 12B, 12C, and 12D show substrate concentration results from simulating threonine synthesis according to various embodiments; and

FIG. 13 shows an example computing device suitable for modeling in silico the kinetics of systems of connected biochemical reactions according to various embodiments.

In the appended figures, similar components and/or features can have the same reference label. Further, various components of the same type can be distinguished by following the reference label by a dash and a second label that distinguishes among the similar components. If only the first reference label is used in the specification, the description is applicable to any one of the similar components having the same first reference label irrespective of the second reference label.

### DETAILED DESCRIPTION

The ensuing description provides preferred exemplary embodiments only, and is not intended to limit the scope, applicability or configuration of the disclosure. Rather, the ensuing description of the preferred exemplary embodiments will provide those skilled in the art with an enabling description for implementing various embodiments. It is understood that various changes may be made in the function and arrangement of elements without departing from the spirit and scope as set forth in the appended claims.

Specific details are given in the following description to provide a thorough understanding of the embodiments. However, it will be understood that the embodiments may be practiced without these specific details. For example, circuits, systems, networks, processes, and other components may be shown as components in block diagram form in order not to obscure the embodiments in unnecessary detail. In other instances, well-known circuits, processes, algorithms, structures, and techniques may be shown without unnecessary detail in order to avoid obscuring the embodiments.

Also, it is noted that individual embodiments may be described as a process which is depicted as a flowchart, a flow diagram, a data flow diagram, a structure diagram, or a block diagram. Although a flowchart or diagram may describe the operations as a sequential process, many of the operations may be performed in parallel or concurrently. In addition, the order of the operations may be re-arranged. A process is terminated when its operations are completed, but could have additional steps not included in a figure. A process may correspond to a method, a function, a procedure, a subroutine, a subprogram, etc. When a process corresponds to a function, its termination may correspond to a return of the function to the calling function or the main function.

### I. Introduction

When modeling a system, determining the boundaries of the system is important. A system can be considered a set of interacting components with some behavior of interest. The real system is never fully isolated from its surroundings. Any model of a system includes and excludes certain components from consideration. The boundary reflects this division between what to include and what to include. In a closed system, mass, energy, and information cannot cross the boundary into or out of the system.

Components of the system within a boundary interact with other components within the boundary but not with the world outside the boundary. If the entire system is within the boundary, then the system cannot increase or decrease in mass or energy. Complicated systems, including biological systems, may interact with many other systems and receive mass, energy, or information across the boundary. While these other systems could be modeled and included within the system boundary, model complexity would then increase. A more complicated model does not translate to a more accurate model as increased model complexity likely increases the number of parameters and unknowns that need to be determined to run the model accurately. Another way to allow interactions across the boundary is to set certain characteristics at a constant. For example, a molecule may be assumed to be at steady-state, and the concentration of a molecule may be set at a constant. Such a technique has the benefit of analytical simplicity, but may force components to behave unrealistically. A molecule may have a constant concentration no matter how quickly it is consumed or produced through reactions, contrary to observed behavior. For at least these reasons, a better way to model the interactions of the system with the world outside the boundary is desired.

Some embodiments model certain components at the boundary of the system as being responsive to changes within the system. The rates of change of the components may respond to deviations of the concentrations of the components from target concentrations. The response that may mimic the behavior of a proportional controller. When the concentration of a given component is much lower than the target concentration, the system responds by increasing the production of the component. When the concentration of a given component is much higher than the target concentration, the system responds by decreasing the amount of the component. At concentrations near the target concentration, the system responds with a smaller rate of change than when the deviation is greater. In some embodiments, the rates of change may be saturable; the rates of change may have a maximum limit when the deviation from the target concentration is high and a minimum limit when the deviation from the target concentration is low. These embodiments may increase the accuracy and efficiency of modeling and may avoid unnecessarily increasing the complexity of a model.

As used herein, the terms “substantially,” “approximately” and “about” are defined as being largely but not necessarily wholly what is specified (and include wholly what is specified) as understood by one of ordinary skill in the art. In any disclosed embodiment, the term “substantially,” “approximately,” or “about” may be substituted with “within [a percentage] of” what is specified, where the percentage includes 0.1, 1, 5, and 10 percent. As used herein, when an action is “based on” something, this means the action is based at least in part on at least a part of the something.

Advantageously, these approaches provide a workflow for modeling the kinematic behavior of a system of reactions. The approaches are general enough for any biochemical system, and scale easily in terms of size and/or complexity. The approaches may take advantage of available data and efficient mathematical constructs. The approaches may avoid requiring a number of rate constants and/or concentrations (and the values associated with the rate constants and/or concentrations) that may be required for other approaches. These other rate constants and/or concentrations may be difficult to obtain experimentally or theoretically. Additionally, some embodiments may avoid enlarging a model to include additional systems that decrease the computational efficiency of a model yet may not significantly improve the accuracy of a model. Approaches described herein also may allow for most computations within the model to pertain to the overall reaction of interest rather than including computations for reactions outside the overall reaction. Approaches described herein focus the modeling on reaction or system of interest and are therefore computationally efficient.

### II. Interaction System and Biological System Modeling Techniques

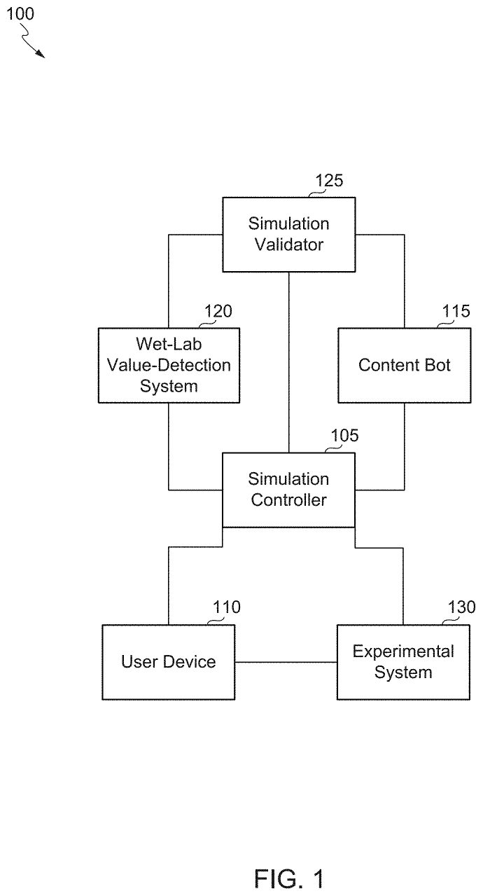

FIG. 1 shows an interaction system 100 for configuring instances or versions of a model and using a simulation to facilitate subsequent experiment configurations (e.g., simulation of a biological system's response to a new demand) according to various embodiments. Each instance of the models may have a combination of modules, perturbations (such as knockouts), and may be built using a particular set of experimental data. In order to facilitate the configuring of a model (e.g., a biological system) and simulate an outcome of the model, the interaction system 100 can include one or more components, each of which can include (for example) one or more servers, one or more computers and/or one or more mobile devices. In some instances, two or more of the components can be included in a same server, same server system, same computer, etc. Interaction system 100 can include one or more networks (e.g., a wired network, a wireless network, the Internet, a local area network, a wide area network, a short-range network, etc.), such that each component in the interaction system 100 can communicate with one or more other components in the interaction system 100.

Interaction system 100 can include a simulation controller 105 that defines, generates, updates and/or executes each of one or more simulations. A simulation can be configured to simulate dynamic progression through states, a time-evolved state of a model of a biological system and/or a steady state based on an iterative module-based assessment. It will be appreciated that identifying a steady-state and/or balanced solution for a module at a given time step need not indicate that a steady-state and/or balanced solution has been, can be or will be identified for the model in general (e.g., as metabolites produced and/or consumed at one module may further be produced and/or consumed at another module that need not be configured for balancing fluxes).

A given model can be used to generate and run any number of simulations. Differing initial conditions and/or differing automatically generated values in stochastic portions of the simulation (e.g., generated using a pseudo-random number generation technique, a stochastic pull from a distribution, etc.) can result in different output results of different simulations. The biological system model can be made up of one or more modules, and during a simulation run, each module is run independently and passes results back up to the biological system model level. More specifically, the biological system (e.g., a whole cell) may be modeled in accordance with a coordinated operation of multiple modules that represent structure(s) and/or function(s) of the biological system. Each module may be defined to execute independently, except that a shared set of state values (e.g., a state vector) maintained at the biological system model level may be used and accessed by multiple modules at each time point.

In some instances, each module of the biological system is configured to advance across iterations (e.g., time points) using one or more physiological and/or physics-based models (e.g., flux balance analysis (FBA), template synthesis, bulk-mass flow analysis, constant non-specific degradation, empirical analysis, etc.). The module-specific iteration processing can further be based on one or more module-specific state values (as determined based on an initial definition for an initial iteration processing or a result of a previous iteration processing for a subsequent iteration processing). The module-specific iteration processing can further be based on one or more parameters defined for the module that are fixed and/or static across iterations across iterations.

Simulation controller 105 can generate simulation configurations using one or more inputs received from a user device 110. For example, simulation controller 105 may generate an interface (or may at least partly define specifications for an interface) that is to be availed and/or transmitted to user device 110 and to include input fields configured to receive inputs that correspond to a selection of (for example) one or more modules to be used for a given biological system model, a model type to be used for each of the one or more modules, one or more parameters that are to be effected by a given module's model and used during execution, and/or one or more initial state-value definitions that are to be used by a given module's model and used during execution. In some instances, the interface identifies a default value for each of one, more or all parameters of the model and for each of one, more or all of the initial-state values of the model and is configured to receive a modification to a subset or all of the parameters and/or initial-state values for which a default value was identified. In some instances, modifying a default initial-state value and/or parameter can correspond to a perturbation of performance of a corresponding module and/or the biological system.

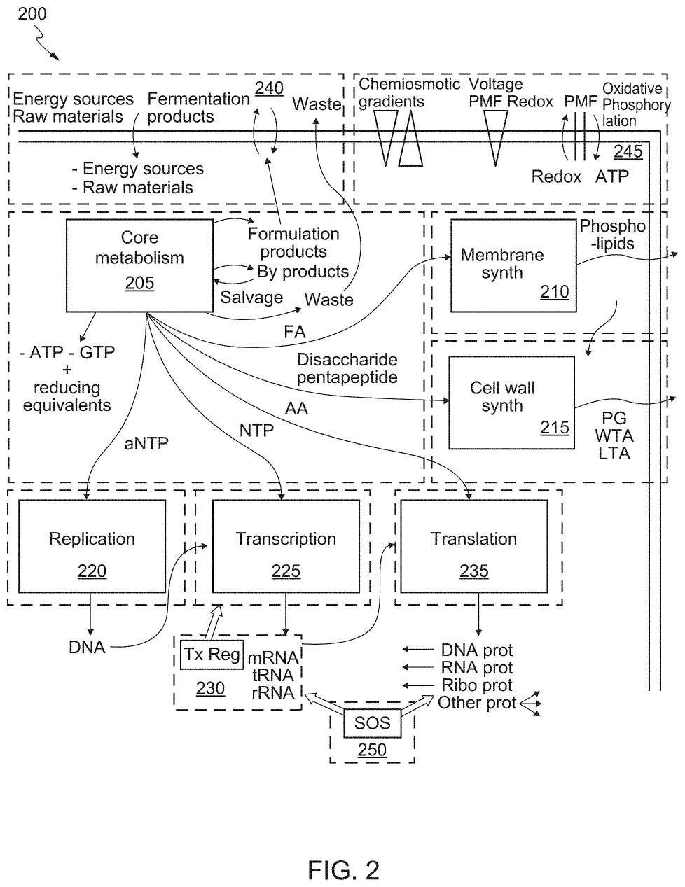

As another example, the interface may further or alternatively be configured to receive an input that corresponds to a selection of one or more default modules and a selection of a model type to be used for each of one or more modules. For example, the interface may include one or more modules (as shown in FIG. 2) representing distinct biological functions in a biological system model, and for each module: a name of the module, a default model type for the module and an option configured to receive a selection of another model type for the module (e.g., that identifies one or more other model types that can be selected for the module).

Default structure of a simulation (e.g., corresponding to default modules, default parameters, default initial-state values and/or default model selections) can be determined based on detected internal or external content and/or based on lab results (e.g., results from physical experiments). The content can include (for example) online, remote and/or local content that is collected by a content bot 115. Content bot 115 can (for example) include a crawler that performs a focused crawling and/or focused browsing (for example) the Internet, a part of the Internet, one or more pre-identified websites, a remote (e.g., cloud-based) storage system, a part of a remote storage system, a local storage system and/or a part of a local storage system. The crawling can be performed in accordance with one or more crawling policies and/or one or more queries that corresponds to one or more modules and/or models (e.g., where each query includes a variable name, representation or description and/or a cellular-function name, representation or description).

The lab results can be received from a wet-lab value detection system 120, which can be configured to trigger performance of one or more investigations (e.g., physical experiments) to detect and/or measure data corresponding to an initial-state value and/or data corresponding to a characteristic or parameter of a biological system. Wet-lab value-detection system 120 can transmit one or more results of the investigation(s) back to simulation controller 105, which may thereafter determine and/or define a default initial-state value or parameter or a possible modification thereof based on the result(s).

Interaction system 100 further includes a simulation validator 125, which can be configured to validate performance of a simulation. The validation may be performed based on pre-identified indications as to how a biological system functions normally and/or given one or more perturbations. Such indications can be defined based on content collected from content bot 115 and/or results from wet-lab value-detection system 120. The data used to validate the simulation may include (for example) one or more balanced values, one or more values indicative of cell dynamics, one or more steady-state values, one or more intermediate values and/or one or more time-course statistics. Simulation validator 125 may return a performance result that includes (for example) a number, category, cluster or binary indicator to simulation controller 105. Simulation controller 105 may use the result to determine (for example) whether a given simulation configuration is suitable for use (e.g., in which case it may be selectable in an interface).

After a simulation is configured with definitions and/or selections of modules, module-specific models, parameters and/or initial-state values, simulation controller 105 can execute the simulation (e.g., in response to receiving an instruction from user device 110 to execute the simulation). The simulation execution can produce one or more simulation results, which may include (for example) one or more balanced values, kinetic values, etc. For example, the simulation can identify a solution for a set of reaction-corresponding stoichiometric equations using linear algebra, such that production and consumption of metabolites represented in the equations is balanced. Notably, this balance may be specific to a given module and need not be achieved for all metabolites produced or consumed by reactions for a given module (e.g., as a non-zero net production or consumption of one or more boundary metabolites may be predefined and/or a target result for a module). Simulation controller 105 can transmit the results (e.g., via an interface) to user device 110.

In some instances, the results can be used to trigger and/or define a subsequent experiment. For example, simulation controller 105 may determine whether a given predefined condition is satisfied based on the results and, if so, may transmit simulation-specific data (e.g., indicating one or more initial-state values, parameters, mutations corresponding to simulation definitions, etc.) to an experimental system 130. The transmission may be indicative of and/or include an instruction to perform an experiment that corresponds to the simulation.

As another example, upon receiving simulation results from simulation controller 105, user device 110 can present an interface that includes some or all of the results and an input component configured to receive input corresponding to an instruction to perform an experiment that corresponds to the simulation. Upon receiving a selection at the input component, user device 110 may transmit data corresponding to the simulation to experimental system 130. After performing a requested experiment, experimental system 130 may return one or more results to simulation controller 105 and/or user device 110.

FIG. 2 shows an illustrative representation of given biological system model 200. The overall modeling strategy includes partitioning the biological system model 200 into modules that can be modeled separately, using a methodology and level of detail appropriate to and/or selected for each module. The partitioning and level of detail for each module can be selected based on (for example) the experiments or simulations that are to be run by the model (e.g., the questions trying to be solved by the model). The selection may be made by the modeler and/or computing system (e.g., the interaction system 100 described with respect to FIG. 1). For example, a user working through an interface of an integrated development environment, a script, and/or an automated system may be implemented to select one or more modules and select a model type to be used for each of one or more modules to ultimately generate the biological system model 200. Additionally or alternatively, the partitioning can be customized and depend on an assessment of the biological functions defined for the initial high-level data set. For example, a separate module may be defined to represent each of the following biological functions: core metabolism 205, membrane synthesis 210, cell-wall synthesis 215, DNA replication 220, transcription 225, transcription regulation 230, translation 235, RNA salvage (not shown), protein and RNA maturation, protein salvage (not shown), transmembrane transport 240 (including electron chain, oxidative phosphorylation, redox, and pH interconversion activity 245), signal transduction (not shown), stress response and growth rate regulation 250, cell division, chemotaxis (not shown), and cell-cell signaling (not shown).

Biological system model 200 can include at least one module that handles core metabolism 205. One possible core metabolic module uses an FBA model, which takes its general shape from standalone FBA, but includes modifications that account for interactions of the core metabolic module with other modules. Each of one, more or all other modules may have their own production and consumption of some of the same molecules within the FBA network, as described in further detail herein. However, as should be understood to those of ordinary skill in the art, an FBA model does not have to be incorporated into the overall biological system model 200 in order for every simulation to work. Instead, various types of models can be used for the modules (e.g., core metabolism 205, membrane synthesis 210, cell-wall synthesis 215, etc.) so long as the type of models can be configured to read values from the state vector and return a list of changes that should be made to the state vector.

For one exemplary instantiation of biological system model 200, core metabolism 205, membrane synthesis 210, and cell-wall synthesis 215 may be encompassed as a single FBA problem, whereas DNA replication 220, transcription 225, transcription regulation 230, and translation 235 may be isolated from the rest of the metabolic network. Meanwhile, transcription 225 and translation 235 may use a template synthesis model, and DNA replication 220 may use a bulk mass-flow model. Transcription regulation 230 may be empirical and static. Optionally, RNA salvage may be modeled using constant non-specific degradation, polymerized DNA, RNA, and protein levels may be determined by the intrinsic rates of the processes that produce them, and the remainder of the components are provided as inputs or parameters of the model.

For another exemplary instantiation of biological system model 200, core metabolism 205 may be encompassed as a single FBA problem. The balance of internal metabolite pools and the supply of building blocks for other processes may be maintained by core metabolism 205. DNA replication 220, transcription 225, transcription regulation 230, and translation 235 may then be isolated from the rest of the metabolic network. Membrane biosynthesis 210 and cell-wall synthesis 215 may be modeled by substrate- and catalyst-driven kinetics. Import and export rates and all exchange with the environment may be driven by the kinetics of membrane transport. Transcription 225 and translation 235 may use a template synthesis model, and DNA replication 220 may use a bulk mass-flow model. Transcription regulation 230 may be empirical and static. Optionally, RNA salvage may be modeled using representations of constant non-specific degradation, while polymerized DNA, RNA, and protein levels may be determined by the intrinsic rates of the processes that produce them, and the remainder of the components for the biological system can be provided as inputs or parameters of the model.

For another exemplary instantiation of biological system model 200, core metabolism 205 may be encompassed as an FBA problem, whereas one or more of membrane synthesis 210, cell-wall synthesis 215, DNA replication 220, transcription 225, transcription regulation 230, and translation 235 can be isolated from the rest of the metabolic network. The balance of internal metabolite pools and the supply of building blocks for other processes may be maintained by core metabolism 205. Membrane biosynthesis 210 and cell-wall synthesis 215 may be modeled by substrate and catalyst driven kinetics. Import and export rates, and all exchange with the environment may be driven by the kinetics of membrane transport. Redox balance, pH, and chemiosmotic gradients may be maintained explicitly. DNA replication 220, transcription 225 and translation 235 may use models based on initiation, elongation, and termination, Transcription regulation 230 may be pattern driven. Stress response and growth rate regulation 250 may be modeled using feedback control mechanisms. Optionally, RNA salvage may be modeled using constant non-specific degradation, while polymerized DNA, RNA, and protein levels may be determined by the intrinsic rates of the processes that produce them, and the remainder of the components for the biological system can be provided as inputs or parameters of the model.

While the biological system model 200 has been described at some length and with some particularity with respect to several described modules, combinations of modules, and simulation techniques, it is not intended that the biological system model 200 be limited to any such particular module configuration or particular embodiment. Instead, it should be understood that the described embodiments are provided as examples of modules, combinations of modules, and simulation techniques, and the modules, combinations of modules, and simulation techniques are to be construed with the broadest sense to include variations of modules, combinations of modules, and simulation techniques listed above, as well as other modules, combinations of modules, and simulation techniques configurations that could be constructed using a methodology and level of detail appropriate to each module and the biological system model 200.

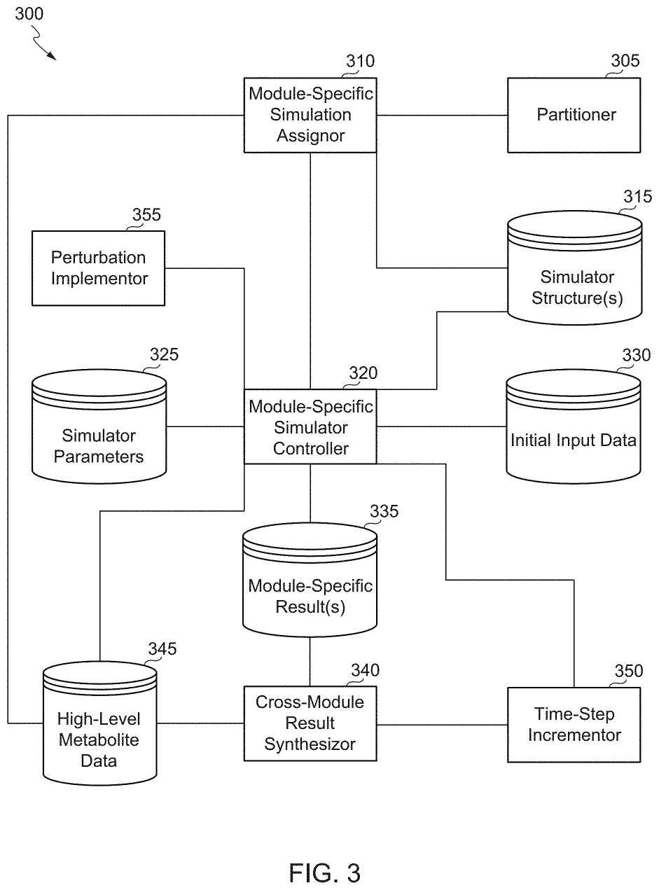

FIG. 3 shows a simulation controller 300 that dynamically integrates results generated by different types of models configured by an integrated development environment (e.g., the interaction system 100 described with respect to FIG. 1) to simulate higher-level states and reactions of a biological system model (e.g., biological system model 200 as described with respect to FIG. 2) according to various embodiments. A partitioner 305 that can identify one or more modules to potentially use for a simulation. In some instances, the modules are identified to correspond to distinct biological functions or physiological processes within a biological system model. Nonetheless, at least one module (e.g., a core module) may address in more detail or cover a larger set of biological functions (e.g., correspond to a core level of physiology across the biological system such as general metabolism of the biological system), whereas at least one other module (e.g., a non-core module) may address in less detail or cover a smaller set biological function (e.g., correspond to transcription and/or translation).

A module-specific simulation assignor 310 may assign, to each module, a simulation type. The simulation type can be selected from amongst one or more types that are associated with the module and/or corresponding physiological process. The one or more types may differ with regard to (for example) a degree of detail to which a physiological process is modeled and/or how the process is modeled. For example, the one or more types may include a simulation using a metabolism-integrated model (e.g., in which specific end products are added to an objective function of a metabolism-based model), substrate- and/or catalyst-drive model using kinetic parameters and reactions, and/or higher-order structure model. A structure for each simulation type (e.g., that indicates how the simulation is to be performed and/or program code) is included in a simulator structure data store 315. Simulator structure data store 315 can further store an association between each simulation type and one or more modules for which the simulation type is associated and is permitted for selection for use.

A module-specific simulator controller 320 can identify, for each module, one or more simulation parameters and an input data set. The simulation parameters may be retrieved from a local data store (e.g., a simulator parameters data store 325) or from a remote source. Each of one or more of the simulation parameters may have been identified based on (for example) user input, a data-fitting technique and/or remote content. The parameter(s), once selected, may be fixed across time-step iterations.

At an initial time step, the input data set can include one or more initial input values, which may be retrieved from a local data store (e.g., an initial input data store 330) or from a remote source. Each of one or more of the initial input values may have been identified based on (for example) user input, a data-fitting technique and/or remote content. With respect to each subsequent time step, the input data set can include (for example) one or more results from a previous iteration of the module and/or one or more high-level results (e.g., cumulative or integrated results) generated from a previous iteration of the multi-module simulation. For example, a module-specific results data store 335 may store each of one, more or all results generated by the assigned simulation for each of one, more or all past time steps, and at least one of the stored results associated with a preceding time step (e.g., most recent time step) can be retrieved.

Upon identifying the input data set and parameters, module-specific simulator controller 320 can run the simulation assigned to the module. Execution of module-specific simulations may be performed concurrently, in parallel and/or using different resources (e.g., different processors, different memory and/or different devices). Results of the simulation run can be stored in module-specific results data store 335.

After results have been generated for each module, a cross-module result synthesizor 340 can access the module-specific results (from one or more module-specific results data stores or direct data availing) and synthesize the results to update high-level data such as a state vector (e.g., stored in a high-level metabolite data store 345). For example, a set of results generated by different modules but relating to a same variable may be identified. The results may be integrated by (for example) summing variable changes as indicated across the results (e.g., potentially with the implementation of one or more caps pertaining to a summed change or to a value of a variable after the summed change is effected). In some instances, a hierarchy is used, such that a result from one module (if available or if another condition is met) is to be exclusively used and a result from another module is to otherwise be used.

Upon synthesizing the results, a time-step incrementor 350 can increment a time step to a next time step so long as the simulation has not completed. It may be determined that the simulation is complete when (for example) processing for a predefined number of time steps has been performed, a particular result is detected (e.g., indicating that a target cell growth has occurred or that a cell has died) or steady state has been reached (e.g., as indicated by values for one or more predefined types of results differing by less than a predefined threshold amount across time steps). When the time step is incremented, module-specific simulator controller 320 can, for each module, collect a new input data set and run the assigned simulation. When the simulation is complete, an output can be generated to include one or more module-specific results, some or all high-level data and/or processed versions thereof. For example, the output may include time-course data for each of one or more metabolites, growth of the biological system over a time period (e.g., as identified by a ratio of availability values of one or more particular metabolites at a final time step as compared to availability values at an initial time step) and/or a growth rate. The output can be transmitted to another device (e.g., to be presented using a browser or other application) and/or presented locally.

Multi-module simulation controller 300 can also include a perturbation implementor 355. Perturbation implementor 355 can facilitate presentation of an interface on a user device. The interface can identify various types of perturbations (e.g., mutations). Perturbation implementor 355 may facilitate the presentation by transmitting data (e.g., HTTP data) to a user device, such that the interface can be presented online. Perturbation implementor 355 can detect a selection that corresponds to a particular perturbation and can send an indication to module-specific simulator controller 320. Module-specific simulator controller 320 can use functional gene data to determine how the mutation affects one or more metabolites and/or one or more simulated processes. A structure of a simulator, one or more simulator parameters and/or one or more initial-input values may then be adjusted in accordance was the perturbation's effects. Thus, multi-module simulation controller 300 can generate output that is indicative of how the perturbation affects (for example) physiological processes and/or growth of the biological system.

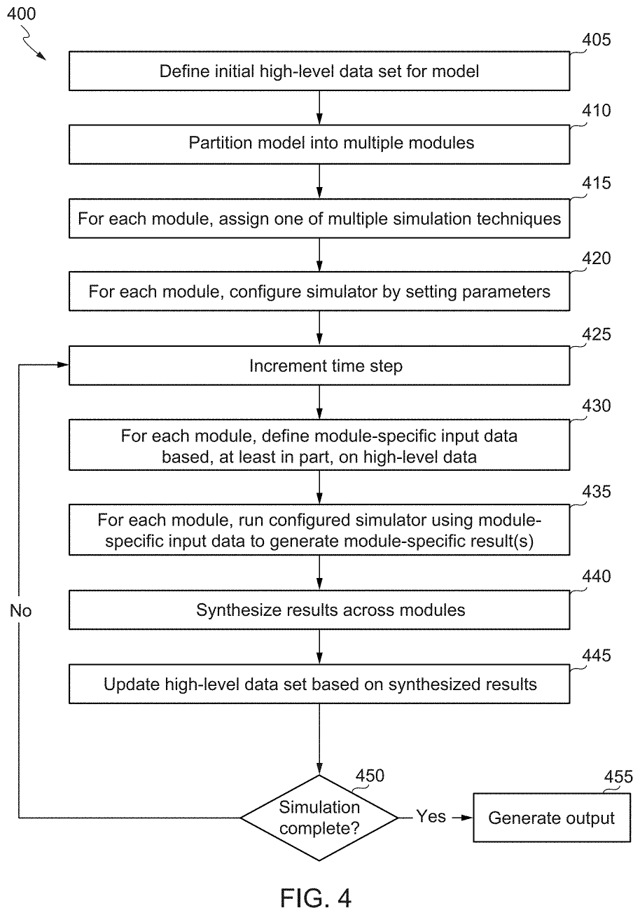

FIG. 4 shows a process 400 for dynamically synthesizing results generated by multiple simulators to simulate higher-level results according to various embodiments. In some embodiments, the processes depicted in process 400 are implemented by the interaction system 100 of FIG. 1, and discussed with respect to the simulation controller 300 of FIG. 4. Process 400 begins at block 405 at which an initial high-level data set is defined for a biological system model. The initial high-level data set can identify (for example) variables, which may be referred to as the state of the biological system model or the state of the simulation, and these variables may be structured as a data structure (e.g., a state vector) and updated throughout a simulation run. In some instances, the variables include an initial availability of each of a set of molecules such as metabolites. The initial availability may be defined based on (for example) a default value, user input, data extracted from content (e.g., online content, remote content or local content that pertains to the molecules), etc. In some instances, the initial availability is determined based on whether any perturbation was identified (e.g., via user input) for a given simulation. If a perturbation was identified, the initial availability may be determined based on a particular perturbation that was identified and by using (for example) a look-up table to determine for which molecule(s) the perturbation affects an availability value and characteristics of such effect.

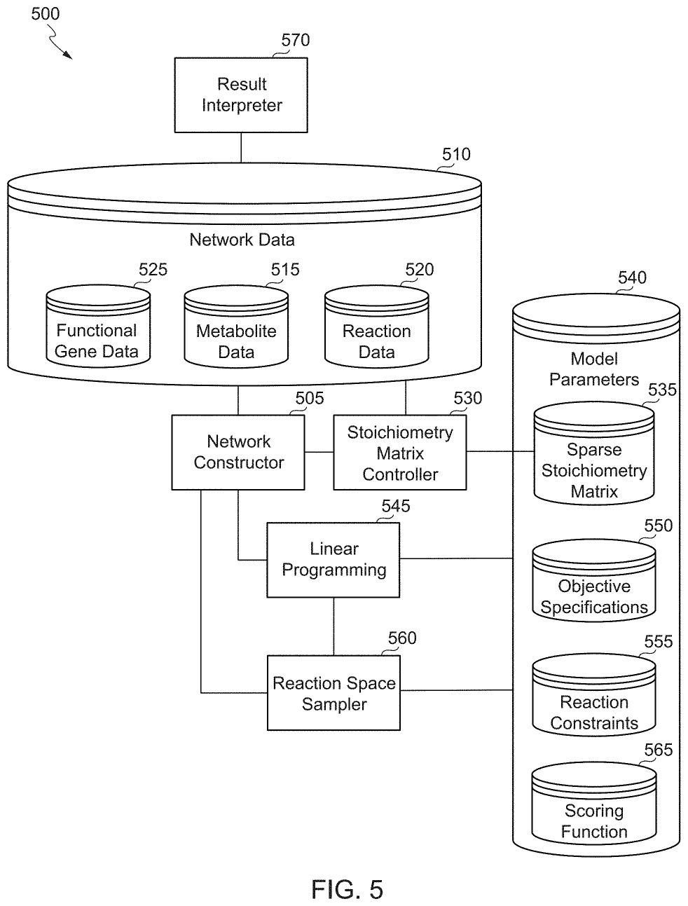

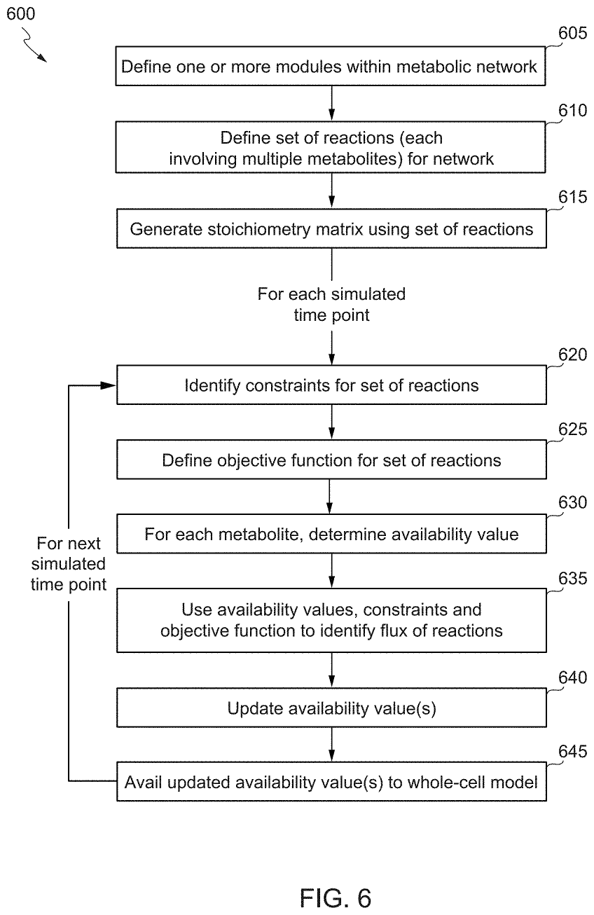

At block 410, a biological system model (e.g., a whole cell model) is partitioned into multiple modules. The partitioning can depend on metabolite dependencies and/or biological-functioning assessment. For example, a separate module may be defined to represent each of the following biological functions: core metabolism, membrane synthesis, cell-wall synthesis, DNA replication, transcription, transcription regulation, translation, RNA salvage, protein and RNA maturation, protein salvage, transmembrane transport (including electron chain, oxidative phosphorylation, redox, and pH interconversion activity), signal transduction, stress response and growth rate regulation (SOS), cell division, chemotaxis, and cell-cell signaling, as discussed in further detail with respect to FIG. 2. In some instances, two or more of these functions may be represented in a core module that models cell composition and growth using a single model. Particular cellular functioning need not be explicitly modeled and instead dynamics of end products of the particular cellular functioning may be modeled. For example, a core module may use a flux-based analysis or a simulation technique as described herein (e.g., in relation to FIG. 5 or FIG. 6).

In some instances, the partitioning may be performed based on user input and/or one or more default configurations. For example, an interface may be presented that identifies each potential separate module (e.g., an interface may be presented via simulation controller 105 as described with respect to FIG. 1). A default configuration may be to integrate the module into a core module (e.g., a core metabolism module) unless a contrary input is received or to perform a simulation using modeling specific to the module unless a contrary input is received. For example, an interface may be configured to receive one or more selections of modules that are to be excluded from a core module and to then integrate each other module into the core module.

At block 415, for each module, one or more simulation techniques are assigned to the module. A simulation technique may include a model type. In some instances, a simulation technique that is assigned to a core module includes a flux-based analysis or other simulation technique, as described herein. In some instances, a simulation technique includes a mechanistic model, a kinetic model, a partial kinetic model, a substrate- and/or catalyst-driven model, and/or a structural model. The simulation technique may be assigned based on (for example) user input and/or one or more predefined default selections. For example, for each non-core module, a default selection may be predefined that represents particular functioning of the module, and for each core module, a default selection may be predefined that simulates dynamics of metabolites across a simulated time period. An interface may identify, for each module, the default selection along with one or more other simulation techniques that are associated with the module (e.g., with the association(s) being based on stored data and/or a predefined configuration). User input may then indicate that an alternative simulation technique is to be used for one or more modules.

At block 420, for each module, a simulator is configured by setting parameters and variables. The parameters (e.g., numeric values) may correspond to inputs to be used in the simulation technique assigned to the module and that are not changed across time steps of the simulation. The particular parameters may be determined based on (for example) stored data, content, a communication from another system and/or user input. The one or more module-specific or cross-module variables (e.g., identifying an initial availability of one or more metabolites) may correspond to inputs to be used in the simulation technique assigned to the module and may be changed across time steps of the simulation. For example, a parameter may be determined for a simulator that sets a minimum viable pH in the cytoplasm (below which the cell dies), and a variable may be identified that describes a current pH in the cytoplasm. The variable (current pH) might change throughout the simulation; however, the parameter (the minimum possible pH) would not change and remains fixed. An initial value of the pH variable may be identified, e.g., the value at the start of the simulation may be set in block 405 or if it is module specific then it may be set in block 420, and like the minimum pH parameter this would be used as an input into the simulation. The values of variables and parameters are both inputs, but the distinction is that variables can change from their initial values, and parameters are fixed throughout the simulation run.

At block 425, a time step is incremented, which can initially begin a given simulation. At block 430, for each module, module-specific input data is defined at least in part on the high-level data. More specifically, a high-level data structure may identify, for each of a set of molecules (e.g., metabolites), an availability value. Each availability value may initially be set to an initial availability value, which may thereafter be updated based on processing results from each module that relates to the molecule. For a given module, at each time step, a current availability value can be retrieved from the data structure for each molecule that pertains to the simulation technique assigned to the module. The module-specific input data may further include one or more lower-level values that are independent from processing of any other module. For example, one or more variables may only pertain to processing of a given module, such that the module-specific input data may further include an initial value or past output value that particularly and exclusively relates to the module.

At block 435, for each module, the configured simulator assigned to the module is run using the module-specific input data to generate one or more module-specific results. The one or more module-specific results may include (for example) one or more updated molecule availability values and/or a change in one or more availability values relative to corresponding values in the input data.

At block 440, results can be synthesized across modules. The synthesis may include summing differences across modules. For example, if a first module's results indicate that an availability of a given molecule is to be increased by 5 units and a second module's results indicate that an availability of the given metabolite is to be decreased by 3 units, a net change may be calculated as being an increase in 2 units. The net change can then be added to a corresponding availability value for the molecule that was used for the processing associated with the current time step and returned as a list of changes that should be made to the state vector. One or more limits may be applied to a change (e.g., to disallow changes across time steps that exceed a predefined threshold) and/or to a value (e.g., to disallow negative availability values and instead set the value to zero).

At block 445, the high-level data set is updated based on the synthesized results. The update can include adding data to a data structure such as a state vector from which one or more modules retrieve high-level data. The added data can include the synthesized results in association with an identifier of a current time step. Thus, the data structure can retain data indicating how an availability of a metabolite changed over time steps. It will be appreciated that alternatively the update can include replacing current high-level data with the synthesized data.

At block 450, it is determined whether the simulation is complete. The determination may be based on a number of time steps assessed, a degree to which data (e.g., high-level data) is changing across time steps, a determination as to whether a steady state has been reached, whether one or more simulated biological events (e.g., cell division or cell death) have been detected, etc. If the simulation is not complete, process 400 returns to block 425.

If the simulation is complete, process 400 continues to block 455, at which an output is generated. The output may include some or all of the high-level data and/or some or all of the module-specific results. For example, the output may include final availability values that correspond to a set of metabolites and/or a time course that indicates a change in the availability of each of one or more metabolites over the simulated time period. The output may be presented at a local device and/or transmitted to another device (e.g., for presentation).

FIG. 5 shows a module-specific simulation controller 500 to simulate states and reactions of modules configured by an integrated development environment (e.g., the interaction system 100 described with respect to FIG. 1) according to various embodiments. A network constructor 505 can be configured to use a model to simulate actions performed by a module of a biological system model (e.g., biological system model 200 as described with respect to FIG. 2). In some instances, the model is flux balance analysis, and/or the model is configured to solve for updated state values based on a set of equations that represent concentration changes in the network (e.g., a metabolic network). As should be understood to those of ordinary skill in the art, a biological system model such as a whole cell model does not have to include an FBA module. For example, from the framework described herein, biological processes such as core metabolism may be modeled that is completely different from FBA. In such an instance, part or all of the description and drawings pertaining to FIGS. 5 and 6 that is specific to FBA (e.g., objective functions, constraints, and linear programming) may not be relevant to that particular instantiation of the model or to simulations run with that model. However, many of the components and techniques described with respect to FIGS. 5 and 6 could be applied to simulate states and reactions of modules implemented by other models. For example, any module can read values from the state vector and return an indication of one or more changes that should be made to the state vector. The FBA module (if it's even present in a particular instantiation of the model) may read and return more values than any other model, but a module modeled with FBA need not be handled by the simulation controller 300 any differently from other modules and/or models described herein.

Network constructor 505 can access a set of network data (e.g., parameters and variables) stored in a network data store 510 to define the model. Metabolite data 515 can identify each metabolite of a metabolome. As used herein, a “metabolite” is any substance that is a product of metabolic action or that is involved in a metabolic process including (for example) each compound input into a metabolic reaction, each compound produced by a metabolic reaction, each enzyme associated with a metabolic reaction, and each cofactor associated with a metabolic reaction. The metabolite data 515 may include for each metabolite (for example) one or more of the following: the name of the metabolite, a description, neutral formula, charged formula, charge, spatial compartment of the biological system and/or module of the model, and identifier such as PubChem ID. Further, metabolite data 515 can identify an initial state value (e.g., an initial concentration and/or number of discrete instances) for each metabolite.

Reaction data 520 can identify each reaction (e.g., each metabolic reaction) associated with the model. For example, a reaction can indicate that one or more first metabolites is transformed into one or more second metabolites. The reaction need not identify one-to-one relationships. For example, multiple metabolites may be defined as reaction inputs and/or multiple metabolites may be defined as reaction outputs. The reaction data 520 may include for each reaction (for example) one or more of the following: the name of the reaction, a reaction description, the reaction formula, a gene-reaction association, genes, proteins, spatial compartment of the biological system and/or module of the model, and reaction direction. Further, the reaction data 520 can identify, for each metabolite of the reaction, a quantity of the metabolite, which may reflect the relative input-output quantities of the involved metabolites. For example, a reaction may indicate that two first metabolites and one second metabolite are input into a reaction and that two third metabolites are outputs of the reaction. The reaction data 520 can further identify an enzyme and/or cofactor that is required for the reaction to occur.

Functional gene data 525 can identify genes and relationships between genes, proteins, and reactions, which combined provide a biochemically, genetically, and genomically structured knowledge base or matrix. Functional gene data 525 may include (for example) one or more of the following: chromosome sequence data, the location, length, direction and essentiality of each gene, genomic sequence data, the organization and promoter of transcription units, expression and degradation rate of each RNA transcript, the specific folding and maturation pathway of RNA and protein species, the subunit composition of each macromolecular complex, and the binding sites and footprint of DNA-binding proteins. Network constructor 505 can use functional gene data and the availability of proteins encoded by those genes to update reaction constraints. One exemplary technique by which genomic data can be associated with reaction data is evaluating Gene-Protein-Reaction expressions (GPR), which associate reactions with specific genes that triggered the formation of one or more specific proteins. Typically a GPR takes the form (Gene A AND Gene B) to indicate that the products of genes A and B are protein sub-units that assemble to form a complete protein and therefore the absence of either would result in deletion of the reaction. On the other hand, if the GPR is (Gene A OR Gene B) it implies that the products of genes A and B are isozymes (i.e., each of two or more enzymes with identical function but different structure) and therefore absence of one may not result in deletion of the reaction. Therefore, it is possible to evaluate the effect of single or multiple gene deletions by evaluation of the GPR as a Boolean expression. If the GPR evaluates to false, the reaction is constrained to zero in the model.

A stoichiometry matrix controller 530 can use reaction data 520 to generate a stoichiometry matrix 535. Along a first dimension of the matrix, different compounds (e.g., different metabolites) are represented. Along a second dimension of the matrix, different reactions are represented. Thus, a given cell within the matrix relates to a particular compound and a particular reaction. A value of that cell is set to 0 if the compound is not involved in the reaction, a positive value if the compound is one produced by the reaction and a negative value if the compound is one consumed by the reaction. The value itself corresponds to a coefficient of the reaction indicating a quantity of the compound that is produced or consumed relative to other compound consumption or production involved in the reaction.

Because frequently relatively few reactions correspond to a given compound, stoichiometry matrix 535 can be a sparse stoichiometry matrix. Stoichiometry matrix 535 can be part of a set of model parameters (stored in a model-parameter data store 540) used to execute a module.

One or more modules may be configured to use linear programming 545 to identify a set of compound quantities that correspond to balancing fluxes identified in reactions represented in stoichiometry matrix 535. Specifically, an equation can be defined whereby the product of stoichiometry matrix 535 and a vector representing a quantity for each of some of the compound quantities is set to zero. (It will be appreciated that the reactions may further include quantities for one or more boundary metabolites, for which production and consumption need not be balanced.) There are frequently multiple solutions to this problem. Therefore, an objective function is defined, and a particular solution that corresponds to a maximum or minimum objective function is selected as the solution. The objective function can be defined as the product between a transposed vector of objective weights and a vector representing the quantity for each compound. Notably, the transposed vector may have a length that is equal to the first dimension of stoichiometry matrix 535, given that multiple reactions may relate to a same compound.

The objective weights may be determined based on objective specifications 550, which may (for example) identify one or more reaction-produced compounds that are to be maximized. For example, the objective weights can be of particular proportions of compounds that correspond to biomass, such that producing compounds having those proportions corresponds to supporting growth of the biological system.

Each reaction may (but need not) be associated with one or more of a set of reaction constraints 555. A reaction constraint may (for example) constrain a flux through the reaction and/or enforce limits on the quantity of one or more compounds consumed by the reaction and/or one or more compounds produced by the reaction.

In some instances, linear programming 545 uses stoichiometry matrix 535 and reaction constraints 555 to identify multiple solutions, each complying with the constraints. When multiple solutions are identified, objective specifications 550 can be used to select from amongst the potential solutions. However, in some instances, no solution is identified that complies with stoichiometry matrix 535 and reaction constraints 555 and/or the only solution that complies with the matrix and constraints is not to proceed with any reaction.

A solution can include one in which, for each of a set of metabolites, a consumption of the metabolite is equal to a production of the metabolite. That is not to say that this balance must be achieved for each metabolite, as a set of reactions involve one or more “boundary metabolites” for which this balance is not achieved. For example, glucose can be consumed at a given rate, and/or acetate can be produced at a given rate.

Reaction data 520 may further identify an objective function that identifies a target product (e.g., representing cell growth rate) that is to be maximized. The objective function can identify particular ratios of multiple reactant metabolites that must be available to produce the product. Strictly enforcing the objective function may result in simulating no growth if a single metabolite is not produced. An alternative approach is to define one or more objective functions configured such that production of each of multiple target reactant metabolites that relate to the target product is to be maximized. A higher level whole-cell model can evaluate the production of multiple target reactant metabolites to determine whether to and/or an extent to which to simulate growth. For example, depending on which target reactant metabolite(s) are not produced, the whole-cell model may nonetheless simulate cell growth, simulate cell growth at a reduced rate, simulate no growth, simulate unhealthy or impaired growth or simulate cell death.

For example, a reaction space can be defined based on stoichiometry matrix 535 and reaction constraints 555. The space may have as many dimensions as there are reactions. Each dimension can be restricted to include only integer values that extend along a range constrained by any applicable constraint in reaction constraints 555. A reaction space sampler 560 can then determine, for each of some or all of the points within the reaction space, a cumulative quantity of each metabolite that would be produced based on the associated reactions. Reaction space sampler 560 can compare these quantities to those in the objective vector (e.g., by determining an extent to which proportions of compounds are consistent).

In these instances, a scoring function 565 can indicate how to score each comparison. For example if proportions of each of two potential solutions differ from the objective proportions by 2, but one potential solution differs by 2 for a single compound and another by 1 for each of two compounds, scoring function 565 can be configured to differentially score these instances. For example, different weights may be applied to different compounds, such that differences that affect a first compound are more heavily penalized than differences that affect a second compound. As another example, scoring function 565 may indicate whether a score is to be calculated by (for example) summing all compound-specific (e.g., weighted) differences, summing an absolute value of all compound-specific (e.g., weighted) differences, summing a square of all compound-specific (e.g., weighted) differences, etc. Reaction space sampler 560 can then identify a solution as corresponding to reaction coefficients that are associated with a highest score across the reaction space.

Network constructor 505 can receive results from each of linear programming 545 and/or reaction space sampler 560. In some instances, linear programming 545 can further avail its results to reaction space sampler 560. When a balanced solution is identified by linear programming 545, reaction space sampler 560 need not sample the reaction space and need not avail reaction-space results to network constructor 505.

Network constructor 505 can identify a solution as corresponding to one identified by linear programming 545 when a balanced solution is identified and as a highest-score potential solution identified by reaction space sampler 560 otherwise. The solution can then indicate the compounds produced by and consumed by the reactions performed in accordance with the solution-indicated flux. Network constructor 505 can update metabolite data 515 based on this production and consumption.

In some instances, a solution is identified for each of a set of time points rather than only identifying one final solution. The iterative time-based approach may be useful when module-specific simulation controller 500 is but one of a set of simulation controllers and metabolite data 515 is influenced by the performance of other modules. For example, metabolite data 515 may be shared across modules or may be defined to be a copy of at least part of a cross-module metabolite data set at each time point. The updates to the metabolites performed by network constructor 505 may then be one of multiple updates. For example, an update by network constructor 505 may indicate that a quantity of a specific metabolite is to increase by four, while a result from another module indicates that a quantity of the specific metabolite is to decrease by two. Then the metabolite may change by a net of +2 for the next time iteration.

A results interpreter 570 can generate one or more results based on the updated metabolite data 515. For example, a result may characterize a degree of growth between an initial state and a steady state or final time point. The degree of growth may be determined based on a ratio between values of one or more metabolites at a current or final time point relative to corresponding values at an initial (or previous) time point. The one or more metabolites may correspond to (for example) those identified in an objective function as corresponding to biomass growth. As another example, a result may characterize a time course of growth. For example, a result may identify a time required for metabolite changes that correspond to a representation of a double in growth or a time constant determined based on a fit to values of one or more time series of metabolite values. The result(s) may be output (e.g., locally presented or transmitted to a remote device, such as a user device). The output can facilitate a presentation of an interface that indicates one or more simulation characteristics (e.g., one or more default values in terms of initial-state values or reaction data and/or one or more effected perturbations).

Operation of module-specific simulation controller 500 can be influenced by particular simulated perturbations of the whole cell. For example, each perturbation may correspond to a particular type of genetic mutation. The perturbation may have been identified based on detecting user input (e.g., a selection and/or text input received via an interface) that defines the perturbation. One exemplary type of perturbation is a gene mutation. An effect of the perturbation may be determined based on functional gene data (e.g., to determine how an availability of one or more metabolites is affected). High-level metabolite data, simulator parameters and/or high-level constraints may then be accordingly set, constrained and/or defined based on the perturbation. This high-level perturbation can thus then influence operation of one or more lower level modules.

FIG. 6 shows a process 600 for using a simulator to generate metabolite time-course data according to various embodiments. In some embodiments, the processes depicted in process 600 are implemented by the interaction system 100 of FIG. 1, and discussed with respect to the module-specific simulation controller 500 of FIG. 5. Process 600 begins at block 605, at which a one or more modules within a metabolic network (e.g., of a biological system) are defined. The module(s) can be defined based on which parts of the network exhibit relative functional independence and/or correspond to substantial independence in terms of biological activity. In some instances, a default is to define each part of a cell as part of a core module unless a different module corresponding to particular types of actions and/or cell components is defined.

At block 610, a set of reactions is defined for the network. In some instances, the set of reactions are defined for the module (or each module) that corresponds to the default model type. The set of reactions can indicate how various molecules such as metabolites are consumed and produced through part of all of a life cycle of a biological system. Each reaction thus identifies one or more metabolites that are consumed, one or more metabolites that are produced and, for each consumed and produced metabolite, a coefficient (which may be set to equal one) indicating a relative amount that is consumed or produced. The reaction may further include an identification of one or more enzymes, one or my cofactors and/or one or more environmental characteristics that are required for the reaction to occur and/or that otherwise affects a probability of the reaction occurring or a property of the reaction. The reactions may be identified based on (for example) online or local digital content (e.g., from one or more scientific papers or databases) and/or results from one or more wet-lab experiments.

At block 615, a stoichiometry matrix is generated using the set of reactions. Each matrix cell within the matrix can correspond to a particular metabolite and a particular reaction. The value of the cell may reflect a coefficient of the particular metabolite within the particular reaction (as indicated in the reaction) and may be set to zero if it is not involved in the reaction. In some instances, metadata is further generated that indicates, for each of one or more reactions, any enzyme, co-factor and/or environmental condition required for the reaction to occur.

At block 620, one or more constraints are identified for the set of reactions. In some instances, identifying the constraints may include identifying values for one or more parameters. For example, for each of one or more or all of the set of reactions, a constraint may include a flux lower bound and/or a flux upper bound to limit a flux, a quantity of a consumed or produced metabolite, a kinetic constant, a rate of production or decay of a component such as RNA transcript, an enzyme concentration or activity, a compartment size, and/or a concentration of an external metabolite. The constraint(s) may be identified based on (for example) user input, online or local data, one or more communications from a wet-lab system, and/or learned from statistical inference.

At block 625, an objective function is defined for the set of reactions. The objective function may identify what is to be maximized and/or what is to be minimized while identifying a solution. The objective function may (for example) identify a metabolite that is produced by one or more reactions or a combination of metabolites that is produced by one or more reactions. The combination may identify proportions of the metabolites. However, the objective function can have a number of limitations and may fail to reflect supply and demand within the other modules. Thus, in some instances, a limited objective function can be constructed to include a set of target values for each molecule within the metabolic network. The target values can incorporate intrinsic-rate parameters, supply rates of molecules, the consumption rates of molecules, and the molecule concentrations into a measurement of target concentrations of the molecule given supply, demand, and an “on-hand” concentration of each molecule, which represents the concentration of a molecule immediately available to a reaction pathway. The target values may be calculated and incorporated into the objective function to produce the limited objective function. This may be in the form of calculating an absolute difference between the target value and the proportional flux contribution of each molecule. This may be in the form of scaling the proportional flux contribution of each molecule. This may be in the form of adding to the proportional flux contribution of each molecule. Any other mathematical modification of the proportional flux contribution of each molecule that adjusts this value by the target value may be used. The target values may be positive or negative. For purposes of unit conversion, so that target values can be included in the objective function and compared to the flux values, the target values may be constructed as rates.

At block 630, for each metabolite related to the set of reactions, an availability value is determined. For an initial value, the value may be identified based on (for example) user input, digital content and/or communication from another system. Subsequent values may be retrieved from a local or remote data object that maintains centralized availability values for the set of metabolites.

At block 635, the availability values, constraints and objective function are used to determine the flux of one, more or all of the set of reactions. The flux(es) may indicate a number of times that each of one, more or all of the reactions were performed in a simulation in accordance with the availability values, constraints and objective function. The flux(es) may be determined based on a flux-balance-analysis model. In some instances, the flux(es) may be determined based on a sampling of all or part of an input space representing different flux combinations and scoring each input-space using a scoring function.

At block 640, a centralized availability value of one or more metabolites is updated based on the determined flux(es). More specifically, for each metabolite, a cumulative change in the metabolite's availability may be identified based on the cumulative consumption and cumulative production of the metabolite across the flux-adjusted set of reactions. The centralized availability value of the metabolite can then be incremented and/or decremented accordingly.

In some instances, at least one the one or more modules defined at block 605 are to be associated with a model that does not depend on (for example) a stoichiometry matrix and/or flux based analysis and/or that is based on physiological modeling. One or more modules based on one or more different types of models can also, at each time point, identify a change in metabolite availability values, and such changes can also be used to update a local or remote data object with centralized availability values. With respect to each metabolite, updates in availability values may be summed to identify a total change and/or updated availability value. In some instances, limits are set with respect to a maximum change that may be effected across subsequent time steps and/or a maximum or minimum availability value for a metabolite.

At block 645, availability data is availed to a higher-level model. State vectors can then be updated based on data from multiple modules.

Some or all of blocks 620-645 may be repeated for each of multiple simulated time points in a simulation. Thus, at each time point, constraints can be updated based on state-vector information (e.g., representing availability of catalysts), an objective function can be defined (e.g., which may change across time points based on a configuration of a higher level objective), updated metabolite availability values can be determined, updated reaction fluxes can be identified, and further updated availability values can be determined. In some instances, a predefined number of simulated time points are to be evaluated and/or simulated time points corresponding to a predefined cumulative time-elapsing period are to be evaluated. In some instances, a subsequent simulated time point is to be evaluated until a predefined condition is satisfied. For example, a predefined condition may indicate that metabolite values for a current simulated time point are the same or substantially similar as compared to a preceding simulated time point or a preceding simulated time period.

With regard to a repeated iteration of block 630, it will be appreciated that an availability value determined for a given metabolite need not be equal to the corresponding updated availability value from the previous iteration of block 640 and/or the sum of the previously determined availability value adjusted by the identified flux pertaining to the metabolite. Rather, a processing of the previous time point with respect one or more other modules may have also resulted in a change in the metabolite availability, and/or a higher level constraint and/or processing may influence the availability. Thus, the availability value for a given metabolite determined at block 630 for a current time point may be equal to the availability value determined at block 630 for a preceding time point plus the cumulative updates to the availability value across modules, with any limits imposed.

While not shown in process 600, one or more variables can be output (e.g., transmitted to a user device). The variable(s) may include final values (e.g., availability values after all iterations have been performed), time-course values, high-level values and/or module-specific values. For example, the availability data may include, for each of one, more or all metabolites: an availability value (e.g., a final availability value) and/or a time course of the availability value. In some instances, the availability data is output with reference availability data. For example, when part or all of the processing performed to calculate the availability values was associated with a perturbation, the reference availability data may be associated with an unperturbed state. In some instances, a processed version of the availability data is output. For example, a comparison of availability values for particular metabolites across time points may be used to generate one or more growth metrics (e.g., a growth magnitude or rate), which may be output. Outputting the availability data can include (for example) locally presenting the availability data and/or transmitting the availability data to another device.

### III. Boundaries in Modeled Systems

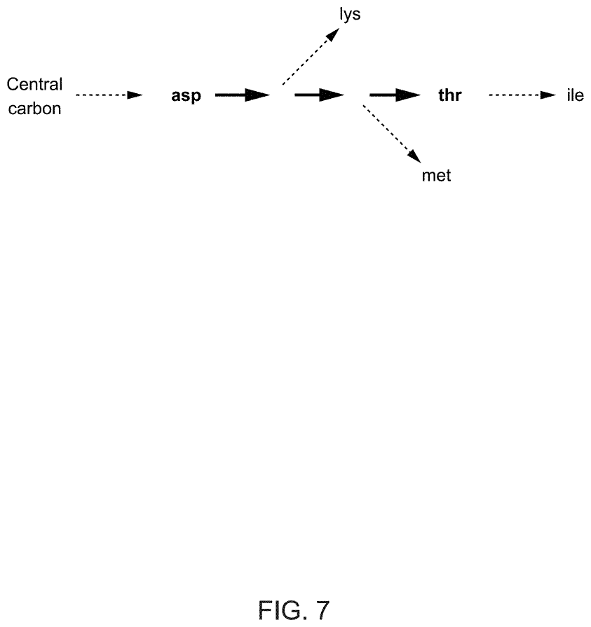

As a specific example of a system to be modeled, consider the production of the amino acid threonine in FIG. 7. The linear pathway from aspartate (asp) to threonine (thr) includes five steps, which can be modeled in the kinetic modeling framework. However, to understand how the pathway will behave in vivo, both this linear pathway and how the pathway connects to the rest of metabolism should be considered.

Intermediates between aspartate and threonine are the starting points for synthesizing lysine (lys) and methionine (met). Threonine itself is the starting point to synthesize isoleucine (ile). The input, aspartate, must also be synthesized. This synthesis takes carbon from glycolysis and the TCA cycle, as well as nitrogen from glutamate. The pathway from aspartate to threonine consumes ATP and NADPH. All of these amino acids are consumed by translation.

To model this system faithfully then becomes an exercise in defining the boundary of the system to be modeled. In the name of completeness, all steps of lysine, methionine, and isoleucine synthesis can be included in a model. This makes the model more complicated, encompassing more unknowns. Expanding the model to include the additional synthesis steps may give us better insight into the interactions of these various pathways. This expanded model, however, has still not included all of central carbon (and nitrogen) metabolism, ATP and NADPH production and consumption, and translation. Even if it were feasible to model all of these processes in full kinetic detail, this further expanded model takes focus away from the pathway of actual interest (production of threonine from aspartate). A boundary to include the parts of the system should be drawn without unreasonably expanding the model to include too many processes that have only indirect effects on the system (e.g, a process that affects the concentration of a component in the main enzymatic reaction without being in the main enzymatic reaction). Additionally, boundaries should be drawn so that the modeled behavior in the system reflects observed behavior.

A. Boundaries in Enzymology

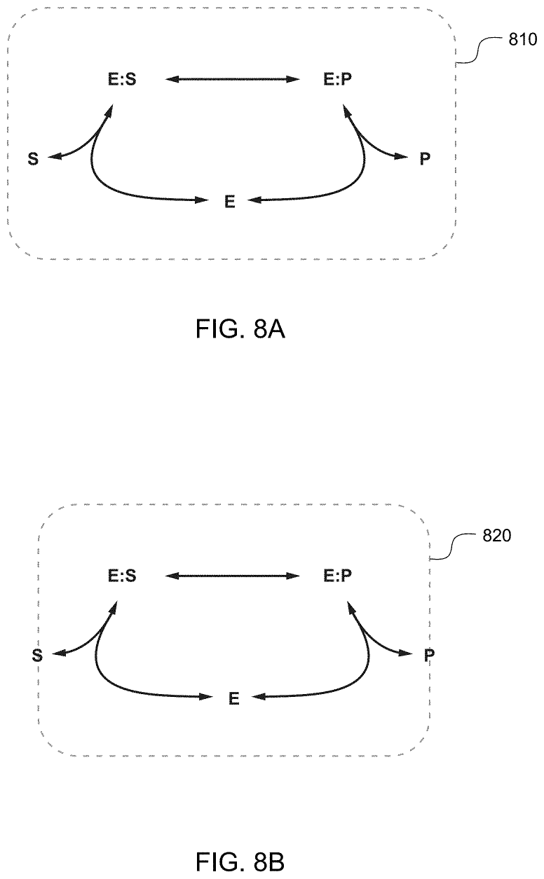

FIG. 8A shows an illustration of an enzymology assay. “S” represents the substrate, “E” represents the enzyme, “P” represents the product, “E:S” represents the enzyme bound to the substrate, and “E:P” represents the enzyme bound to the product. Dashed line 810 illustrates the system boundary. The substrate, product, and all enzyme forms are inside the boundary. Accordingly, the substrate, product, and all enzyme forms can interact with each other but not with anything outside the system boundary. As a result, neither the substrate nor the product can leave the system. Over time, the concentrations of the substrate and the product reach equilibrium. This figure represents the nature of an assay in a closed tube. The system is considered isolated. No mass, energy, or information crosses the system boundary.

FIG. 8B shows an illustration of an enzymology assay. “S” represents the substrate, “E” represents the enzyme, “P” represents the product, “E:S” represents the enzyme bound to the substrate, and “E:P” represents the enzyme bound to the product. Dashed line 820 illustrates the system boundary. All enzyme forms are inside the boundary. Unlike FIG. 8A, the system boundary crosses the substrate and the product. The substrate and the product may still interact with the enzyme forms. In addition, because they cross the system boundary, the substrate and the product may be affected by external influences. As a result, mass and energy may enter and exit the system through the substrate and product. A model describing the enzymology assay then should specify how external influences impact the system through the substrate and product.

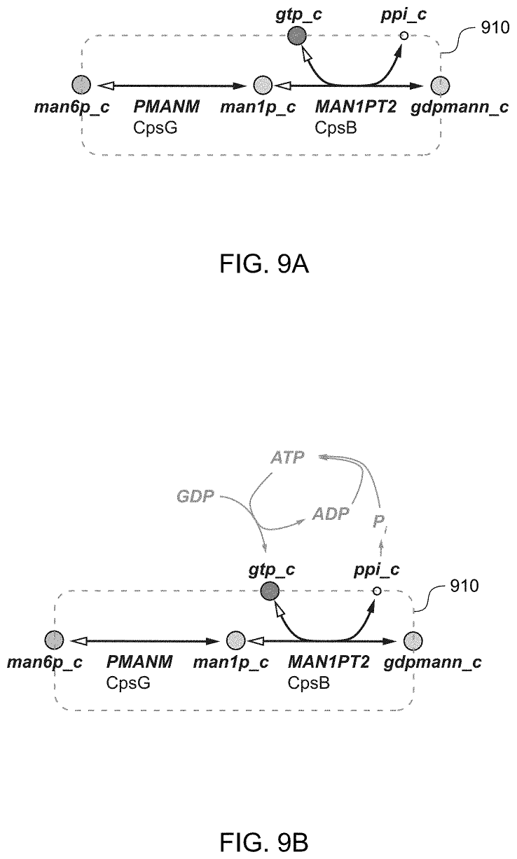

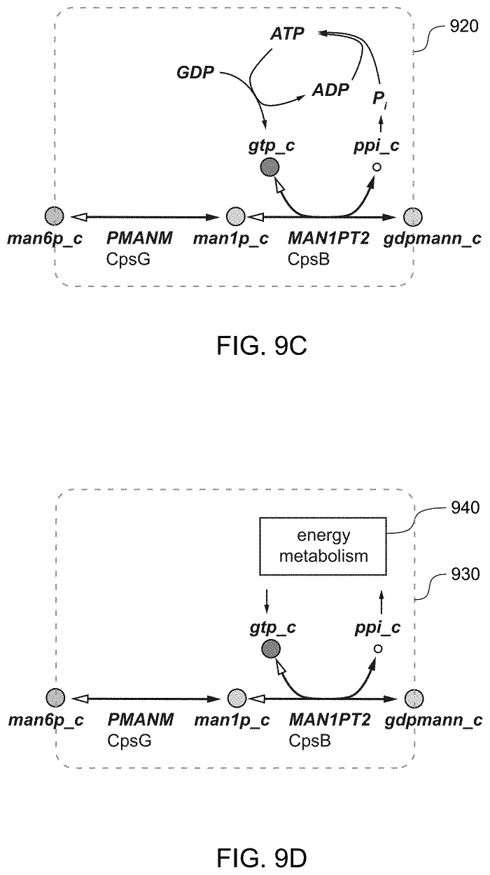

FIG. 9A shows a specific example of an enzymology assay, the pathway from mannose-6P to GDP-mannose. Mannose-6P is represented by “man6p_c”. Mannose-1P is represented by “man1p_c”. GDP-mannose is represented by “gdpmann_c”. GTP is represented by “gtp_c”. Pyrophosphate is represented by “PPᵢ”. Two enzymes are involved in the process: phosphomannomutase (CpsG) (“PMANM”) and mannose-1-phosphate guanylyltransferase (CpsB) (“MAN1PT2”). System boundary 910 crosses mannose-1P (i.e., the substrate), GDP-mannose (i.e., the product), and GTP and pyrophosphate. In this manner, these molecules can be produced or consumed by the actions outside the system, as expected in a real biological system.

FIG. 9B illustrates the pathway from mannose-6P to GDP-mannose and the energy metabolism for producing GTP. The production of GTP and consumption of PPi are external forces that affect the pathway from mannose-6P to GDP-mannose.

FIG. 9C illustrates expansion of the system boundary to include energy metabolism with the pathway from mannose-6P to GDP-mannose. Dashed line 920 represents the expanded system boundary. As a result, GDP, ATP, ADP, and phosphate (Pᵢ) are within the model and interact with other molecules in the model. At the same time, GDP, ATP, ADP, and phosphate are produced and consumed in other reaction pathways, so modeling them as completely internal to the system is not realistic. The system boundary can then be expanded farther to consider the effects on GDP, ATP, ADP, and phosphate, but such a system boundary expansion will likely bring in more molecules that are affected by yet other reaction pathways. Expanding the system boundary to include GDP, ATP, ADP, and phosphate has not resolved boundary issues faced in FIG. 9A.

FIG. 9D illustrates modeling the energy metabolism as a simplified process with the pathway from mannose-6P to GDP-mannose. Dashed line 930 is the system boundary and drawn around an energy metabolism process 940. Instead of including the individual molecules, the model may include an energy metabolism process 940 to simulate the production of GTP and the consumption of pyrophosphate. While conceptually this may be a solution, a separate model then must be created for energy metabolism. Such a model may not exist, and a new model may need to be verified against data, distracting from modeling and analysis of the main pathway.

The concentrations of molecules at the boundary can be specified without expanding the system boundary further or adding a sub-model to the system.

B. Static Boundaries

One approach to model the molecules at the system boundary is to treat the concentrations as invariant, through the simple expedient of forcing their rates of change to be zero. Returning to FIG. 7, the system boundary may be drawn to cross at aspartate. The system may include reactions from aspartate to threonine but no reactions upstream of aspartate. If the model system ran in isolation, downstream steps will consume aspartate, i.e. produce a negative

$$\frac{d [ asp ]}{dt} ,$$

which would over time deplete the starting pool of aspartate.

To prevent this, after the other calculations are done,

$$\frac{d [ asp ]}{dt}$$

can be forced to be zero. This is functionally the same as adding in an equal and opposite change, in effect treating upstream reactions as replenishing aspartate at exactly the rate necessary to maintain its observed intracellular concentration.

An invariant boundary as described here behaves as a perfect source—the system can draw as much from the source as needed, and the source will maintain itself at a constant level. The same reasoning applies when

$$\frac{dy}{dt}$$

is a sink, where y is the concentration of a molecule. The kinetics of the system generate an excess of the boundary molecule y instead of consuming it. By forcing

$$\frac{dy}{dt}$$

to zero, we treat the world outside our boundary as consuming any production at exactly the rate necessary to maintain a constant level. This is a perfect sink.

Perfect sources and perfect sinks may be convenient and expedient in a modeling framework but do not reflect real behavior in biological systems. The concentrations of sources and sinks are expected to vary as the molecules are produced and consumed by reactions within the system and outside of the system.

C. Responsive Boundaries

Instead of modeling a boundary as static, the boundaries can be modeled as responsive and less than perfect. A responsive boundary is a boundary that has a rate of change in concentration that varies depending on a load on the boundary. The expected characteristics of a boundary is that the concentration level is maintained under a light load but exhibits the impact of a heavier load. An imperfect source can be depleted; an imperfect sink can back up. The goal is to have the boundaries respond to increasing load in a realistic way, without having to model all of the details of the world beyond the boundary.

Another way to think about this is that within the system boundary of a kinetic model, what matters is the hard concept of concentration. Every reaction is driven by the immediate, current concentrations of all molecules involved. Behavior of the system evolves over time only because these concentrations change, as a result of the reactions themselves. Responsive boundaries represent the more flexible concept of availability, combining a molecule's current concentration with the capacity of the system to replenish or absorb it. For a fully modeled system, availability of a molecule emerges from the dynamics of all of the reactions affecting it. A responsive boundary that captures at least some of these dynamics in a more abstract way provides the benefit of a more detailed model without unnecessarily expanding the scope.

The distinction between concentration and availability further gives us a means to model “off-pathway” effects in a meaningful way. For instance, if a pathway consumes a redox carrier such as NADPH, changes to the redox maintenance machinery may affect the pathway indirectly. With a static boundary, the only way this change can be represented is by raising or lowering the static concentration of NADPH. With a responsive boundary, the same initial concentration of NADPH may be used, but the replenishment rate may be altered. The system then responds by finding a level that draws as much NADPH as it can without dramatically depleting the source.

D. Proportional Controller

One way to model the boundary as responsive is to model the rate of the molecule as a proportional controller. The boundary may include two attributes: a setpoint and a proportional constant, kₚ. The setpoint is the steady-state concentration of the molecule in the absence of any load. The proportional constant is a proportional rate applied in the direction of the setpoint. The rate of change of the molecule is termed an offset. The offset may be defined by the following equation:

offset=kₚ(setpoint−state) (Equation 1).

The state is the instant concentration of the molecule.

The source is replenished (or the sink drained) at a rate scaled proportionally by how far the concentration deviates from its constant load-free state. The higher the proportional factor, the harder it works against any load.

In control theory, a purely proportional controller is considered relatively primitive, because the state must deviate from its setpoint in order to generate an offset. A system with any load can never perfectly reach its target. In the case of real-world, enzymatic systems, this may be an advantageous feature rather than a drawback. In the real-world system, the boundary molecule is expected to be affected by the load being applied; how much it is affected is a reflection of the processes that restore it, as represented by the proportional factor kₚ. The inability of the concentration to perfectly reach its target is unlikely to significantly affect kinetics as a small deviation from the target may not impact the calculated rates.

Another concern about proportional controllers is that they are prone to oscillation, as the corrective force may overshoot the target. In practice, this has not been observed as a problem in test simulations using the kinetic model. In effect, the proportional controller adjusts its offset instantaneously as the state approaches its setpoint and does not overshoot. However, if oscillation were to be observed as a problem in the future, additional measures may be taken to dampen the oscillations. Dampening the oscillations may include modeling the boundary as a proportional-integral (PI) controller or a proportional-integral-derivative (PID) controller. In addition, any term that decreases the magnitude of the offset may dampen the oscillations.

E. Saturable

One more possible extension to this responsive boundary approach is to make the offset rate saturable. The offset may constrained to be between a maximum rate and a minimum rate. This characteristic may be accomplished with a saturation factor, kₛₐₜ. Our calculation then becomes:

$$\begin{matrix}sep = setpoint - state & ( Equation 2 ) \\ offset = \frac{{k}_{p} {k}_{sat} sep}{sep + {k}_{sat}} & ( Equation 3 )\end{matrix}$$

When sep<<kₛₐₜ, the offset reduces to kₚ×sep (linearly related to the instant concentration). Conversely, when sep>>kₛₐₜ, the offset reduces to kₚ×kₛₐₜ (constant, saturated). This saturable behavior tracks well with Michaelis-Menten style saturation kinetics, which are empirically common in biological systems.

F. External Loads

In some embodiments, external processes may impose a non-zero load on the modeled system, across a defined boundary. Returning to the FIG. 7, an example is translation, which imposes an external demand on all of the amino acids in the model system. Expanding the system boundary. to include translation may result in unnecessary complexity. However, being able to capture the basics of external loads may be useful in understanding the system.

A load can be negative (external demand) or positive (external supply). Either type of load should respond to actual availability or concentration. At a minimum, if the quantity becomes limited the load must not be able to drive the quantity past zero.

Mathematically, a demand load such as translation may behave exactly as a saturable sink, and a supply load may be treated as a saturable source. In some models, a molecule that acts as a load (e.g., a source or a sink) may act as a load at multiple microsteps in the model. The effects of multiple loads may be additive, but because they may also be non-linear, the combined effect of multiple loads may not be reduced into a single calculation. Multiple loads, when considered saturable, may be modeled as independent loads at the boundary rather than modeled as a single load. Nevertheless, generalizing the idea that a boundary may be represented by multiple independent saturable sources and sinks may improve the accuracy and usefulness of a model.

### IV. Example Methods

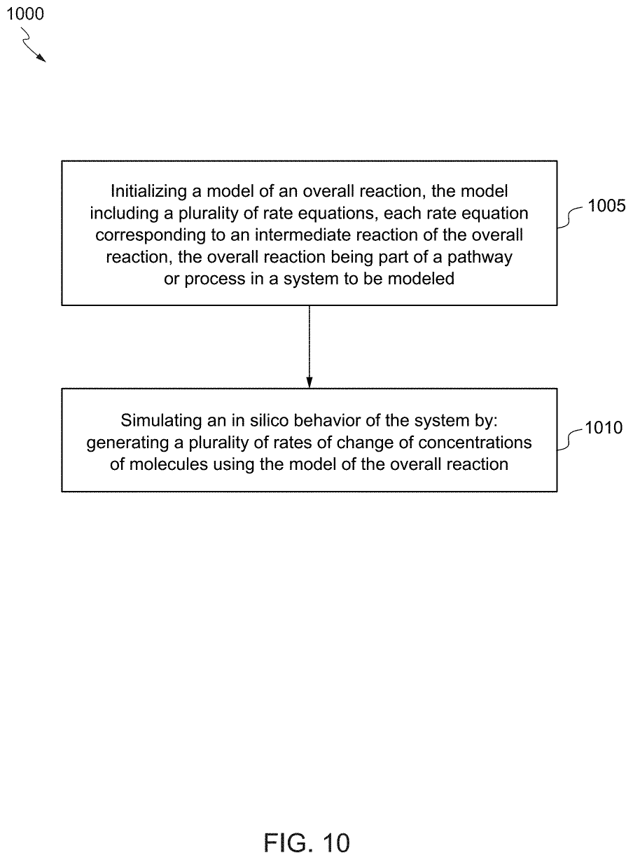

FIG. 10 shows a computer-implemented method 1000 for modeling an overall reaction. The overall reaction may be any reaction disclosed herein. For example, the overall reaction may be an enzymatic reaction or a metabolic reaction. The overall reaction may include a plurality of intermediate reactions. Intermediate reactions may be considered microscopic steps (“microsteps”) of an overall reaction. Examples of the microsteps are any of the individual steps depicted in FIG. 8A, 8B, or 9A-9D. The sum or completion of the intermediate reactions may be the overall reaction. The number of intermediate reactions may be from 2 to 10, from 10 to 50, from 50 to 100, from 100 to 200, from 200 to 500, from 500 to 1,000, or greater than 1,000. The method may apply to any model described herein and models described in U.S. application Ser. No. 16/942,222, filed Jul. 29, 2020, the entire contents of which are incorporated herein by reference for all purposes.

At block 1005, method 1000 may include initializing a model of the overall reaction. The model may include a plurality of rate equations. Each rate equation may correspond to an intermediate reaction of the overall reaction. The overall reaction may be part of a pathway or process in a system to be modeled. The system may include a biological cell, such as E. coli. The pathway may include any metabolic pathway or cycle, such as the citric acid cycle. The system may represent a biological system, including core metabolism, membrane synthesis, cell-wall synthesis, DNA replication, transcription, transcription regulation, translation, RNA salvage, protein and RNA maturation, protein salvage, transmembrane transport (including electron chain, oxidative phosphorylation, redox, and pH interconversion activity), signal transduction, stress response and growth rate regulation (SOS), cell division, chemotaxis, and cell-cell signaling.

The plurality of rate equations may include concentrations of molecules. The molecules may include a first molecule. The plurality of rate equations may include a first rate equation. The first rate equation may correspond to a first intermediate reaction of the overall reaction. The first rate equation may include a concentration of the first molecule. The number of different molecules to have concentrations tracked may total from 2 to 10, from 10 to 50, from 50 to 100, from 100 to 200, from 200 to 500, from 500 to 1,000, or greater than 1,000.

The rate equations may have a form of a forward rate constant k_{fwd} multiplied by a single concentration or two concentrations, and where each reverse rate equation has a form of a reverse rate constant kᵣₑᵥ multiplied by a single concentration or two concentrations. In some embodiments, the concentration or concentrations may be raised to a power, which may be 2, 3, or a non-integer. The rate equations may be determined based on the stoichiometry of the intermediate reaction. In some cases, the rate equations may be based on or adjusted by experimental data or published data. The model of the overall reaction may include a set of ordinary differential equations, where each ordinary differential equation corresponds to a rate equation.

The model may be configured such that the concentration of the first molecule is not increased in the plurality of rate equations other than the first rate equation. For example, the first molecule may be considered as source or a sink and may not be increased or decreased other than at the boundary of the model. A source may be a molecule that may increase in concentration through mechanisms external to the boundary of the system. A sink may be a molecule that may decrease in concentration through mechanisms external to the boundary of the system. In some embodiments, a source may be a molecule that if the concentration was not subject to constraints other than rate equations, the concentration of the molecule would reach 0 at infinite time so long as concentrations of other molecules in the reaction did not reach zero. In some embodiments, a sink may be a molecule that if the concentration was not subject to constraints other than the rate equations, the sink would continually increase as time increased so long as concentrations of other molecules in the reaction did not reach zero.

If the concentration of the first molecule is a source, then the concentration of the first molecule is being primarily decreased in intermediate reactions. Example sources may include the substrate S in FIGS. 8A and 8B and mannose-6P (man6p_c) in FIGS. 9A-9D. As a source, the first molecule may be only consumed as represented by the first rate equation or possibly other rate equations. In some embodiments, the concentration of the first molecule as a source may not increase at all through intermediate reactions. In some embodiments, the concentration of the first molecule as a source may increase through intermediate reactions representing the reverse of the first rate equation and of possibly the other rate equations. The reverse rate constant in these reactions may be much less than the forward rate constant and not including the reverse reaction in the model may not significantly impact the concentrations of the first molecule and/or other molecules in the model.

If the concentration of the first molecule is a sink, then the concentration of the first molecule may not decrease through any intermediate reactions other than a reaction representing the reverse of the first rate equation. Examples of sinks may include the product P in FIGS. 8A and 8B and GDP-mannose (gdpmann_c) in FIGS. 9A-9D. As a sink, the first molecule may be only generated as represented by the first rate equation or possibly other rate equations. In some embodiments, the concentration of the first molecule as a sink may not decrease at all through intermediate reactions. In some embodiments, the concentration of the first molecule as a sink may decrease through intermediate reactions representing the reverse of the first rate equation (and possibly other equations). In some embodiments, the reverse rate constant in these reactions may be much less than the forward rate constant and, not including the reverse reaction in the model, may not significantly impact the concentrations of the first molecule and/or other molecules in the model.

A rate of change of the concentration of the first molecule may be configured to depend on a separation value of the concentration from a setpoint. The rate of change of the concentration of the first molecule may be configured to be proportional to the separation value. The rate of change of the concentration may be represented by:

rate=kₚsep (Equation 4)

where kₚ is a proportional constant and sep is the separation value of the concentration from a setpoint. The separation value may be defined as

sep=setpoint−state (Equation 5)

where setpoint is a specific, predetermined concentration and state is the concentration of the first molecule in a given state (e.g., for a particular time step).

Equations 4 and 5 show that when the setpoint is greater than the state, and the rate of change of the concentration is positive. Equations 4 and 5 show that when the setpoint is less than the state, and the rate of change of the concentration is negative. Equation 4 may representing the rate of change of the concentration (i.e., the offset) as a proportional controller because the offset is proportional to a difference from a setpoint.

The rate of change of the concentration of the first molecule may be set to depend on a saturation constant and a proportional constant. The rate of change of the concentration of the first molecule may be set to approach the product of the saturation constant and the proportional constant as the separation value increases. The rate of change of the concentration of the first molecule may be set to approach the product of the proportion constant and the separation value as the separation value decreases. Including the saturation constant creates limits to the offset response. The saturation constant, the proportional constant, or the setpoint may be adjusted after comparing a generated rate of change of the concentration of the first molecule with a reference rate of change of the concentration of the first molecule. The adjusting of the saturation constant, the proportional constant, or the setpoint may be by a programmer or a computer system, through regression analysis or other machine learning techniques. The rate of change of the concentration (i.e., offset) may be represented by:

$$offset = \frac{{k}_{p} {k}_{sat} sep}{sep + {k}_{sat}} \qquad ( Equation 6 )$$

where kₚ is a proportional constant, kₛₐₜ is a saturation constant, and sep is a separation value.

The offset may have units of a concentration rate, for example, μM s⁻¹. The setpoint may have units of concentration (e.g., μM). The proportional constant, kₚ, may have units of inverse time (e.g., s⁻¹), similar to a decay constant. The saturation constant, kₛₐₜ, may have units of concentration, such as μM. The saturation constant may be similar to Kₘ in Michaelis-Menten kinetics. The product of kₚ and kₛₐₜ may be similar to Vₘₐₓ in Michaelis-Menten kinetics.

While the rate of change of the concentration of the first molecule may be defined using equations as described above, the rate of change may also or alternatively be based on readings from physical systems. For example, a computer-implemented method may including sending information to and receiving information from a physical proportional controller (e.g., an electronic proportional controller, a fly-ball governor, or certain biological systems). The information from the physical proportional controller may include an electrical characteristic (e.g., a current or a voltage) or a physical characteristic (e.g., a displacement or a velocity). Information representing a deviation from a setpoint may be sent to the physical controller, and information received may be the response to the physical controller. The physical controller may be programmed for its own physical setpoint (e.g., a current, voltage, displacement, or velocity), which may represent the concentration setpoint in the model. As a result, a mathematical calculation may not be required in a determination of the rate of change of the concentration of the first molecule.

At block 1010, method 1000 may include simulating an in silico behavior the system. Simulating may be by generating a plurality of rates of change of the concentrations of molecules using the model of the overall reaction. Simulating the behavior of the system may include iteratively calculating concentrations of molecules. For a first iteration, a first concentration of the first molecule may be generated using the first rate equation or may be the concentration at the initial condition. This first concentration may be the value of state in Equation 5. The rate of change of the concentration of the first molecule may be generated using the separation value of the first concentration from the setpoint. A second concentration of the first molecule may be determined using the generated rate of change of the concentration. For a second iteration, the plurality of rates of change of the concentrations of the molecule may be generated using the second concentration of the first molecule in the model of the overall reaction. As a result, the first concentration need not be equal to the concentration at a second time step or iteration.

The simulation may include additional iterations, each representing a time step of the reaction. At each time step, the concentration of the first molecule may be adjusted toward the setpoint, proportional to the difference from the setpoint. In some embodiments, the rate of change may reach saturation rates for large differences or small differences from the setpoint.

Some embodiments may also include systems. The system may include one or more data processors. The system may also include a non-transitory computer readable storage medium containing instructions which, when executed on the one or more data processors, cause the one or more data processors to perform actions including any of the methods described herein.

Some embodiments may also include a computer-program product tangibly embodied in a non-transitory machine-readable storage medium, including instructions configured to cause one or more data processors to perform actions including any of the methods described herein.

After simulating the in silico behavior of the system, a biological system may be engineered or altered based on the simulated behavior. A new gene that may increase the production or activity of an enzyme may be introduced. The expression of an existing gene may be increased or decreased. A particular protein may be engineered to target certain enzymatic properties. These modifications may be introduced in E. coli or other cellular organisms. The behavior of the model may identify certain factors to be adjusted so as to narrow the design space for synthetic biology or metabolic engineering. Time and costs for new biological developments, including new drugs and treatments for disorders, may then be reduced. In some embodiments, after simulating the in silico behavior of the system, an experiment may be run to verify the simulated behavior.

### V. Example Results

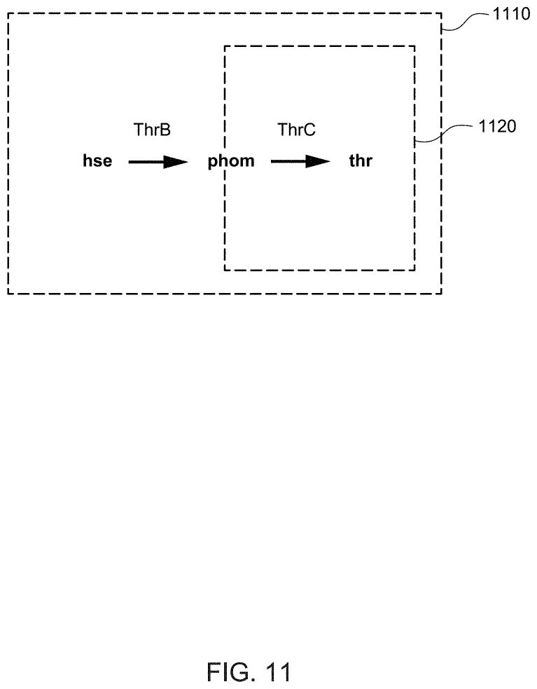

Responsive boundaries are tested using threonine synthesis. The threonine synthesis reaction pathway is illustrated in FIG. 7. FIG. 11 shows the last two steps of threonine synthesis in more detail. Hse is homoserine, ThrB is homoserine kinase, phom is phospho-homoserine, ThrC is threonine synthetase, and thr is threonine. Threonine synthesis is simulated using two system boundaries: system boundary 1110, which includes all substrates and enzymes in the figure in the system, and system boundary 1120, which includes phospho-homoserine at the boundary.

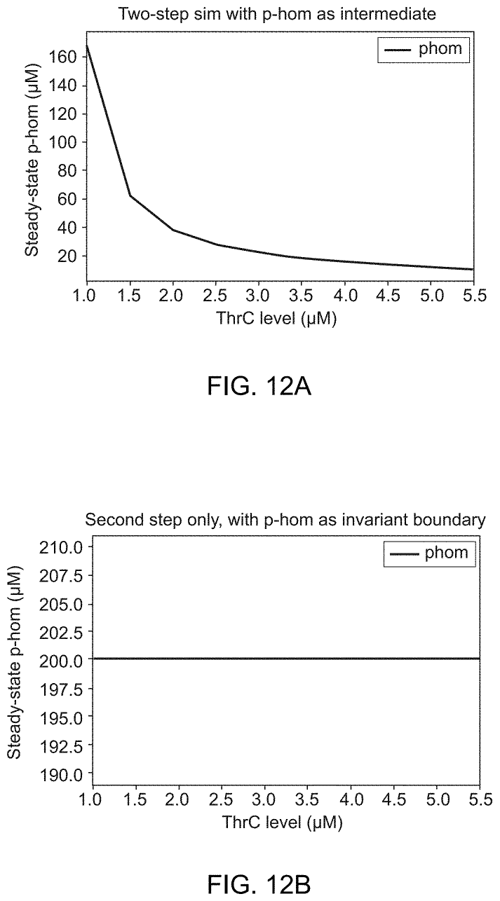

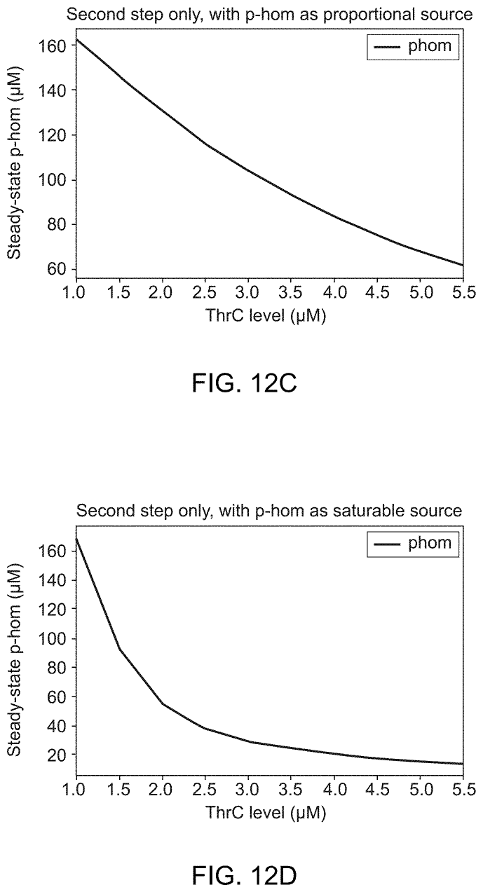

FIG. 12A shows results of simulating the last two steps of threonine synthesis. The system boundary is system boundary 1110. Both reaction steps are simulated over a range of ThrC levels. Phospho-homoserine settles in at a steady-state concentration. The graph in FIG. 12A shows the steady-state concentration of phospho-homoserine in μM on the y-axis and the ThrC level in μM on the x-axis.

FIG. 12B shows results of simulating the last step of threonine synthesis with phospho-homoserine held a constant level. The steady-state concentration of phospho-homoserine in μM is on the y-axis, and the ThrC level in μM on the x-axis. Phospho-homoserine acts as an invariant or static boundary and does not respond to changes in how fast it is consumed by ThrC. FIG. 12B shows a horizontal flat line, which is much different from the curve in FIG. 12A.

FIG. 12C shows results of simulating the last step of threonine synthesis with phospho-homoserine set to respond proportionally to a difference from a setpoint. The steady-state concentration of phospho-homoserine in μM is on the y-axis, and the ThrC level in μM on the x-axis. The response of phospho-homoserine to concentration changes in ThrC is analogous to a proportional controller. The shape of the curve shows that the steady state concentration of phospho-homoserine decreases with increasing ThrC levels. The response of phospho-homoserine to levels of ThrC in FIG. 12C is more similar to FIG. 12A than FIG. 12B is to FIG. 12A.

FIG. 12D shows results of simulating the last step of threonine synthesis with phospho-homoserine set to respond proportionally to a difference from a setpoint with saturability. The steady-state concentration of phospho-homoserine in μM is on the y-axis, and the ThrC level in μM on the x-axis. The response of phospho-homoserine to concentration changes in ThrC is similar in shape to the response in FIG. 12A.

Of the responses in FIGS. 12B, 12C, and 12D, the response of phospho-homoserine in FIG. 12D is the most similar to the response when modeling two reaction steps in FIG. 12A. The example results show that modeling a boundary as proportional and saturable can be used to generate a result similar to that used in a more complicated model with an expanded boundary.

### VI. Example Computing Environment

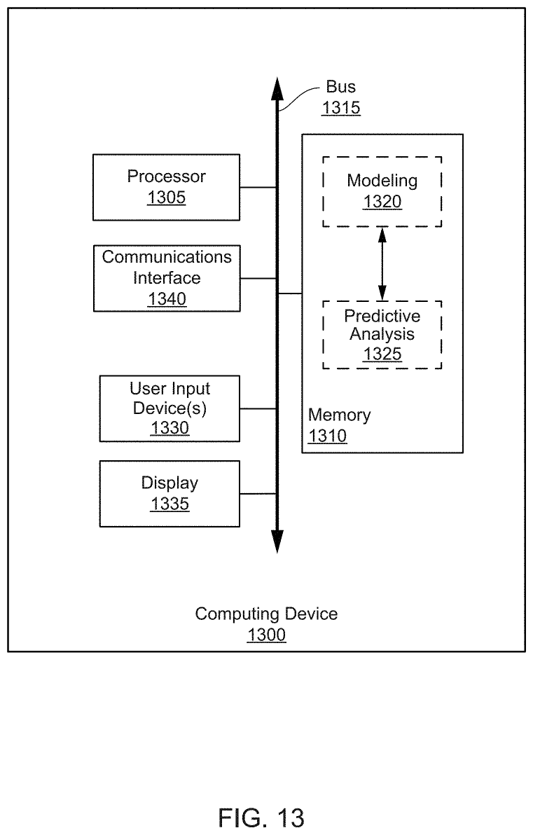

FIG. 13 illustrates an example computing device 1300 suitable for modeling in silico the kinetics of systems of connected biochemical reactions according to this disclosure (e.g., running a module of biological system model 200 described with respect to FIG. 2). The example computing device 1300 includes a processor 1305 which is in communication with the memory 1310 and other components of the computing device 1300 using one or more communications buses 1315. The processor 1305 is configured to execute processor-executable instructions stored in the memory 1310 to perform one or more methods for modeling in silico the kinetics of systems of connected biochemical reactions according to different examples, such as part or all of the example processes 400, 600, and 1000, described herein with respect to FIGS. 1-12. In this example, the memory 1310 stores processor-executable instructions that provide a modeling module 1320 and a predictive analysis module 1325 for one or more reaction pathways, processes, or systems of interest, as discussed herein with respect to FIGS. 1-12.

The modeling module 1320 (e.g., part of the biological system model 200 described with respect to FIG. 2) may be configured to derive a in silico behavior of the system based on the contribution of each component step of the plurality of component steps to the rate of change of the molecules within a pathway, process, or reaction. The predictive analysis module 1325 may be configured to predict a new energy profile, setpoints, constants, and/or parameters, using a prediction model, for the reaction based on the derived in silico behavior of the reaction. Thereafter, the contribution of each component step to the rate of change of the molecules within the pathway, process, or reaction may be applied iteratively over a unit of time back to a state vector. For example, after results have been generated by the modeling module 1320 and the predictive analysis module 1325, a cross-module result synthesizor can access the module-specific results (from one or more module-specific results data stores or direct data availing) and synthesize the results to update high-level data such as a state vector (e.g., stored in a high-level metabolite data store). The state vector may then be used as input to simulate states and reactions of modules configured by an integrated development environment (e.g., the interaction system 100 described with respect to FIG. 1) and/or used by a simulator (e.g., module-specific simulation controller 500 described with respect to FIG. 5) to generate metabolite time-course data according to various embodiments.

The computing device 1300, in this example, also includes one or more user input devices 1330, such as a keyboard, mouse, touchscreen, microphone, etc., to accept user input. The computing device 1300 also includes a display 1335 to provide visual output to a user such as a user interface. The computing device 1300 also includes a communications interface 1340. In some examples, the communications interface 1340 may enable communications using one or more networks, including a local area network (“LAN”); wide area network (“WAN”), such as the Internet; metropolitan area network (“MAN”); point-to-point or peer-to-peer connection; etc. Communication with other devices (e.g., other devices within the interaction system 100 described with respect to FIG. 1) may be accomplished using any suitable networking protocol. For example, one suitable networking protocol may include the Internet Protocol (“IP”), Transmission Control Protocol (“TCP”), User Datagram Protocol (“UDP”), or combinations thereof, such as TCP/IP or UDP/IP.

### VII. Additional Considerations

Specific details are given in the above description to provide a thorough understanding of the embodiments. However, it is understood that the embodiments can be practiced without these specific details. For example, circuits can be shown in block diagrams in order not to obscure the embodiments in unnecessary detail. In other instances, well-known circuits, processes, algorithms, structures, and techniques can be shown without unnecessary detail in order to avoid obscuring the embodiments.

Implementation of the techniques, blocks, steps and means described above can be done in various ways. For example, these techniques, blocks, steps and means can be implemented in hardware, software, or a combination thereof. For a hardware implementation, the processing units can be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments can be described as a process which is depicted as a flowchart, a flow diagram, a data flow diagram, a structure diagram, or a block diagram. Although a flowchart can describe the operations as a sequential process, many of the operations can be performed in parallel or concurrently. In addition, the order of the operations can be re-arranged. A process is terminated when its operations are completed, but could have additional steps not included in the figure. A process can correspond to a method, a function, a procedure, a subroutine, a subprogram, etc. When a process corresponds to a function, its termination corresponds to a return of the function to the calling function or the main function.

Furthermore, embodiments can be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks can be stored in a machine readable medium such as a storage medium. A code segment or machine-executable instruction can represent a procedure, a function, a subprogram, a program, a routine, a subroutine, a module, a software package, a script, a class, or any combination of instructions, data structures, and/or program statements. A code segment can be coupled to another code segment or a hardware circuit by passing and/or receiving information, data, arguments, parameters, and/or memory contents. Information, arguments, parameters, data, etc. can be passed, forwarded, or transmitted via any suitable means including memory sharing, message passing, ticket passing, network transmission, etc.

For a firmware and/or software implementation, the methodologies can be implemented with modules (e.g., procedures, functions, and so on) that perform the functions described herein. Any machine-readable medium tangibly embodying instructions can be used in implementing the methodologies described herein. For example, software codes can be stored in a memory. Memory can be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium”, “storage” or “memory” can represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, wireless channels, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer-implemented method comprising:
initializing a model of an overall reaction, the model including a plurality of rate equations, each rate equation corresponding to an intermediate reaction of the overall reaction, the overall reaction being part of a pathway or process in a system to be modeled, wherein:
the plurality of rate equations comprises concentrations of molecules,
the molecules comprise a first molecule,
the plurality of rate equations comprises a first rate equation,
the first rate equation corresponds to a first intermediate reaction of the overall reaction,
the first rate equation comprises a concentration of the first molecule, and
a rate of change of the concentration of the first molecule is configured to depend on a separation value of the concentration from a setpoint; and
simulating an in silico behavior of the system by:
generating a plurality of rates of change of the concentrations of molecules using the model of the overall reaction.

2. The computer-implemented method of claim 1, wherein:
the rate of change of the concentration of the first molecule is configured to depend on a saturation constant and a proportional constant,
the rate of change of the concentration of the first molecule is configured to approach the product of the saturation constant and the proportional constant as the separation value increases, and
the rate of change of the concentration of the first molecule is configured to approach the product of the proportional constant and the separation value as the separation value decreases.

3. The computer-implemented method of claim 2, wherein:
the saturation constant, the proportional constant, or the setpoint is adjusted after comparing a generated rate of change of the concentration of the first molecule with a reference rate of change of the concentration of the first molecule.

4. The computer-implemented method of claim 1, wherein the model is configured such that the concentration of the first molecule is not increased or not decreased in the plurality of rate equations other than in the first rate equation and a second rate equation corresponding to a second intermediate reaction that is the reverse of the first intermediate reaction.

5. The computer-implemented method of claim 4, wherein the model is configured such that the concentration of the first molecule is not increased in the plurality of rate equations other than the first rate equation.

6. The computer-implemented method of claim 1, wherein the rate of change of the concentration of the first molecule is configured to be proportional to the separation value.

7. The computer-implemented method of claim 1, wherein the rate of change of the concentration is represented by:
$$\frac{{k}_{p} {k}_{sat} sep}{sep + {k}_{sat}}$$
where:
kₚ is a proportional constant,
kₛₐₜ is a saturation constant, and
sep is the separation value.

8. A system comprising:
one or more data processors; and
a non-transitory computer readable storage medium containing instructions which, when executed on the one or more data processors, cause the one or more data processors to perform actions including:
initializing a model of an overall reaction, the model including a plurality of rate equations, each rate equation corresponding to an intermediate reaction of the overall reaction, the overall reaction being part of a pathway or process to be modeled, wherein:
the plurality of rate equations comprises concentrations of molecules,
the molecules comprise a first molecule,
the plurality of rate equations comprises a first rate equation,
the first rate equation corresponds to a first intermediate reaction of the overall reaction,
the first rate equation comprises a concentration of the first molecule, and
a rate of change of the concentration of the first molecule is configured to depend on a separation value of the concentration from a setpoint; and
simulating an in silico behavior of the system by:
generating a plurality of rates of change of the concentrations of molecules using the model of the overall reaction.

9. The system of claim 8, wherein:
the rate of change of the concentration of the first molecule is configured to depend on a saturation constant and a proportional constant,
the rate of change of the concentration of the first molecule is configured to approach the product of the saturation constant and the proportional constant as the separation value increases, and
the rate of change of the concentration of the first molecule is configured to approach the product of the proportional constant and the separation value as the separation value decreases.

10. The system of claim 9, wherein:
the saturation constant, the proportional constant, or the setpoint is adjusted after comparing a generated rate of change of the concentration of the first molecule with a reference rate of change of the concentration of the first molecule.

11. The system of claim 8, wherein the model is configured such that the concentration of the first molecule is not increased or not decreased in the plurality of rate equations other than in the first rate equation and a second rate equation corresponding to a second intermediate reaction that is the reverse of the first intermediate reaction.

12. The system of claim 11, wherein the model is configured such that the concentration of the first molecule is not increased in the plurality of rate equations other than the first rate equation.

13. The system of claim 8, wherein the rate of change of the concentration of the first molecule is configured to be proportional to the separation value.

14. The system of claim 8, wherein the rate of change of the concentration is represented by:
$$\frac{{k}_{p} {k}_{sat} sep}{sep + {k}_{sat}}$$
where:
kₚ is a proportional constant,
kₛₐₜ is a saturation constant, and
sep is the separation value.

15. A computer-program product tangibly embodied in a non-transitory machine-readable storage medium, including instructions configured to cause one or more data processors to perform actions including:
initializing a model of an overall reaction, the model including a plurality of rate equations, each rate equation corresponding to an intermediate reaction of the overall reaction, the overall reaction being part of a pathway or process in a system to be modeled, wherein:
the plurality of rate equations comprises concentrations of molecules,
the molecules comprise a first molecule,
the plurality of rate equations comprises a first rate equation,
the first rate equation corresponds to a first intermediate reaction of the overall reaction,
the first rate equation comprises a concentration of the first molecule, and
a rate of change of the concentration of the first molecule is configured to depend on a separation value of the concentration from a setpoint; and
simulating an in silico behavior of the system by:
generating a plurality of rates of change of the concentrations of molecules using the model of the overall reaction.

16. The computer-program product of claim 15, wherein:
the rate of change of the concentration of the first molecule is configured to depend on a saturation constant and a proportional constant,
the rate of change of the concentration of the first molecule is configured to approach the product of the saturation constant and the proportional constant as the separation value increases, and
the rate of change of the concentration of the first molecule is configured to approach the product of the proportional constant and the separation value as the separation value decreases.

17. The computer-program product of claim 16, wherein:
the saturation constant, the proportional constant, or the setpoint is adjusted after comparing a generated rate of change of the concentration of the first molecule with a reference rate of change of the concentration of the first molecule.

18. The computer-program product of claim 15, wherein the model is configured such that the concentration of the first molecule is not increased or not decreased in the plurality of rate equations other than in the first rate equation and a second rate equation corresponding to a second intermediate reaction that is the reverse of the first intermediate reaction.

19. The computer-program product of claim 18, wherein the model is configured such that the concentration of the first molecule is not increased in the plurality of rate equations other than the first rate equation.

20. The computer-program product of claim 15, wherein the rate of change of the concentration of the first molecule is configured to be proportional to the separation value.
